SEQUENCE LISTING (49 sequences)

SEQ ID NO: 1 - protein, 13 aa, synthetic construct
  VARIANT 1: note = K, R, or H
  VARIANT 2: note = K, R, H, T or L
  VARIANT 3: note = F, Y, W or K
  VARIANT 6: note = R, K, P, H or L
  VARIANT 7: note = K, R, P, H or T
  VARIANT 8: note = P, S or L
  VARIANT 9: note = K or R
  VARIANT 11: note = P, K, R, H or L
  VARIANT 12: note = K, W, R or H
XXXWCXXXXWXXC

SEQ ID NO: 2 - protein, 13 aa, synthetic construct
  VARIANT 2: note = K, R, H, T or L
  VARIANT 3: note = F, Y, W or K
  VARIANT 6: note = R, K, P, H or L
  VARIANT 7: note = K, R, P, H or T
  VARIANT 8: note = P, S or L
  VARIANT 11: note = P, K, R or L
  VARIANT 12: note = K, W or R
KXXWCXXXKWXXC

SEQ ID NO: 3 - protein, 13 aa, synthetic construct
  VARIANT 2: note = K, R or T
  VARIANT 3: note = F or W
  VARIANT 6: note = R, K or P
  VARIANT 7: note = K, R, P, H or T
  VARIANT 8: note = P or S
  VARIANT 11: note = P, K, R or H
  VARIANT 12: note = K, W, R or H
KXXWCXXXKWXXC

SEQ ID NO: 4 - protein, 13 aa, synthetic construct
  VARIANT 2: note = K or R
  VARIANT 3: note = F or W
  VARIANT 6: note = R, K or P
  VARIANT 7: note = K, R or P
  VARIANT 8: note = P or S
  VARIANT 11: note = P, K or R
  VARIANT 12: note = K, W or R
KXXWCXXXKWXXC

SEQ ID NO: 5 - protein, 13 aa, synthetic construct
  VARIANT 1: note = K, R or H
  VARIANT 2: note = K, R, H, T or L
  VARIANT 3: note = F, Y, W or K
  VARIANT 4: note = A, W, P, F or Y
  VARIANT 6: note = R, K, P, H or L
  VARIANT 7: note = K, R, P, H or T
  VARIANT 8: note = P, S or L
  VARIANT 9: note = K or R
  VARIANT 10: note = W, V, F or Y
  VARIANT 11: note = P, K, R, H or L
  VARIANT 12: note = K, W, R or H
XXXXCXXXXXPKC

SEQ ID NO: 6 - protein, 13 aa, synthetic construct
  VARIANT 1: note = K, R or H
  VARIANT 2: note = K, R, H, T or L
  VARIANT 3: note = F, Y, W or K
  VARIANT 4: note = A, W, P or F
  VARIANT 6: note = R, K, P, H or L
  VARIANT 7: note = K, R, P, H or T
  VARIANT 8: note = P, S or L
  VARIANT 10: note = W or V
  VARIANT 11: note = P, K, R or L
  VARIANT 12: note = K, W or R
XXXXCXXXKXXKC

SEQ ID NO: 7 - protein, 13 aa, synthetic construct
  VARIANT 1: note = K, R or H
  VARIANT 2: note = K, R, H or T
  VARIANT 3: note = F, Y, W or K
  VARIANT 4: note = A, W, P or F
  VARIANT 6: note = R, K or P
  VARIANT 7: note = K, R, P, H or T
  VARIANT 8: note = P or S
  VARIANT 10: note = W, V or F
  VARIANT 11: note = P, K or R
  VARIANT 12: note = K or W
KXXXCXXXKXXXC

SEQ ID NO: 8 - protein, 13 aa, synthetic construct
  VARIANT 2: note = K or R
  VARIANT 3: note = F, Y or W
  VARIANT 6: note = R, K or P
  VARIANT 7: note = K, R or P
  VARIANT 8: note = P or S
  VARIANT 10: note = W, V or F
  VARIANT 4: note = A, W, P or F
  VARIANT 11: note = P, K or R
  VARIANT 12: note = K or W
KXXXCXXXKXXXC

SEQ ID NO: 9 - protein, 13 aa, synthetic construct
  MOD_RES 13: note = NH2
KKFACRKPKVPKC

SEQ ID NO: 10 - protein, 13 aa, synthetic construct
  MOD_RES 13: note = NH2
KKFACRKPKWPKC

SEQ ID NO: 11 - protein, 13 aa, synthetic construct
  MOD_RES 13: note = NH2
KKFWCRKPKWPKC

SEQ ID NO: 12 - protein, 13 aa, synthetic construct
  MOD_RES 13: note = NH2
KLFWCRKPKWPKC

SEQ ID NO: 13 - protein, 13 aa, synthetic construct
  MOD_RES 13: note = NH2
KKKWCLKPKWPKC

SEQ ID NO: 14 - protein, 13 aa, synthetic construct
  MOD_RES 13: note = NH2
KKFWCRKLKWPKC

SEQ ID NO: 15 - protein, 13 aa, synthetic construct
  MOD_RES 13: note = NH2
KKFWCRKPKWLKC

SEQ ID NO: 16 - protein, 13 aa, synthetic construct
  MOD_RES 13: note = NH2
KTYPCPPSKVKDC

SEQ ID NO: 17 - protein, 13 aa, synthetic construct
  MOD_RES 13: note = NH2
KTYPCPPSKVKWC

SEQ ID NO: 18 - protein, 13 aa, synthetic construct
  MOD_RES 13: note = NH2
KKYPCPPSKWKWC

SEQ ID NO: 19 - protein, 13 aa, synthetic construct
  MOD_RES 13: note = NH2
KTWPCPPSKWKKC

SEQ ID NO: 20 - protein, 13 aa, synthetic construct
  MOD_RES 13: note = NH2
KKWPCPPSKWKKC

SEQ ID NO: 21 - protein, 13 aa, synthetic construct
  MOD_RES 13: note = NH2
KTYWCPPSKWKDC

SEQ ID NO: 22 - protein, 13 aa, synthetic construct
  MOD_RES 13: note = NH2
KKYWCPPSKWKKC

SEQ ID NO: 23 - protein, 13 aa, synthetic construct
  MOD_RES 13: note = NH2
KKFWCPPSKWKKC

SEQ ID NO: 24 - protein, 13 aa, synthetic construct
  MOD_RES 13: note = NH2
KTYRCPTPKVPNC

SEQ ID NO: 25 - protein, 13 aa, synthetic construct
  MOD_RES 13: note = NH2
KTYRCPTPKWPKC

SEQ ID NO: 26 - protein, 13 aa, synthetic construct
  MOD_RES 13: note = NH2
KKWRCPTPKWPKC

SEQ ID NO: 27 - protein, 13 aa, synthetic construct
  MOD_RES 13: note = NH2
KTWRCPPPKWPKC

SEQ ID NO: 28 - protein, 13 aa, synthetic construct
  MOD_RES 13: note = NH2
KKWRCPPPKWPKC

SEQ ID NO: 29 - protein, 13 aa, synthetic construct
  MOD_RES 13: note = NH2
KKYWCPTPKWPKC

SEQ ID NO: 30 - protein, 13 aa, synthetic construct
  MOD_RES 13: note = NH2
KKFWCPTPKWPKC

SEQ ID NO: 31 - protein, 13 aa, synthetic construct
  MOD_RES 13: note = NH2
KKYPCPHPKWRKC

SEQ ID NO: 32 - protein, 13 aa, synthetic construct
  MOD_RES 13: note = NH2
KKWPCPHPKWRKC

SEQ ID NO: 33 - protein, 13 aa, synthetic construct
  MOD_RES 13: note = NH2
KKWPCPKPKWRKC

SEQ ID NO: 34 - protein, 13 aa, synthetic construct
  MOD_RES 13: note = NH2
KKYWCPHPKWRKC

SEQ ID NO: 35 - protein, 13 aa, synthetic construct
  MOD_RES 13: note = NH2
KKFWCPHPKWRKC

SEQ ID NO: 36 - protein, 13 aa, synthetic construct
  MOD_RES 13: note = NH2
KTYPCPHPKVRDC

SEQ ID NO: 37 - protein, 34 aa, synthetic construct
KKMPCKRRRDCKTYPCPHPKVRDCVKGYCKCVVR

SEQ ID NO: 38 - protein, 32 aa, synthetic construct
TKPCQSDKDCKKFACRKPKVPKCINGFCKCVR

SEQ ID NO: 39 - protein, 26 aa, synthetic construct
SRDCKTYPCPPSKVKDCIKGYCKCVR

SEQ ID NO: 40 - protein, 33 aa, synthetic construct
TKQPCKSRKHCKTYRCPTPKVPNCVNGFCKCVR

SEQ ID NO: 41 - protein, 18 aa, synthetic construct
KDCKKFACRKPKVPKCIN

SEQ ID NO: 42 - protein, 20 aa, synthetic construct
  VARIANT 1: note = K, R, S, H or A
  VARIANT 2: note = K, R, H, W, F or Y
  VARIANT 3: note = K, H, R, W, F or Y
  VARIANT 4: note = K, R or H
  VARIANT 5: note = K, R, H, T or L
  VARIANT 6: note = F, Y, W, H, R or K
  VARIANT 7: note = W, F or Y
  VARIANT 9: note = R, K, P, L or H
  VARIANT 10: note = K, R, P, T or H
  VARIANT 11: note = P, S or L
  VARIANT 12: note = K, R or H
  VARIANT 13: note = W, F or Y
  VARIANT 14: note = P, K, R or L
  VARIANT 15: note = K, W or R
  VARIANT 17: note = I, V, A, K, F, Y or L
  VARIANT 18: note = N, K, R, H, F or Y
  VARIANT 19: note = A, K, F, or Y
  VARIANT 20: note = Y, W or K
XXXXXXXCXXXXXXXCXXGF

SEQ ID NO: 43 - protein, 32 aa, synthetic construct
TKPCAAAAACKKFACRKPKVPKCINGFCKCVR

SEQ ID NO: 44 - protein, 32 aa, synthetic construct
TKPCQSDKDCAAAACRKPKVPKCINGFCKCVR

SEQ ID NO: 45 - protein, 32 aa, synthetic construct
TKPCQSDKDCKKFACAAAAVPKCINGFCKCVR

SEQ ID NO: 46 - protein, 32 aa, synthetic construct
TKPCQSDKDCKKFACRKPKAAACINGFCKCVR

SEQ ID NO: 47 - protein, 32 aa, synthetic construct
TKPCQSDKDCKKFACRKPKVPKCAAAACKCVR

SEQ ID NO: 48 - protein, 10 aa, synthetic construct
KKFACRKPKV

SEQ ID NO: 49 - protein, 6 aa, synthetic construct
KREAEA

(19) United States
(12) Patent Application Publication
(10) Pub. No.: US 2025/0282822 A1
(43) Pub. Date: Sep. 11, 2025

(54) SYNTHETIC ANTIMICROBIAL PEPTIDES

(71) Applicant: Donald Danforth Plant Science Center, ST. LOUIS, MO (US)

(72) Inventors: Dilip M. Shah, Chesterfield, MO (US); Meenakshi Tetorya, St. Louis, MO (US)

(21) Appl. No.: 18/861,412

(22) PCT Filed: May 2, 2023
(86) PCT No.: PCT/US2023/066505

§ 371 Date: Oct. 29, 2024

Related U.S. Application Data
(60) Provisional application No. 63/364,077, filed on May 3, 2022.

(51) Int. Cl. C07K 7/08; A01N 37/46; A01P 1/00; A01P 3/00; C07K 14/00

(52) U.S. Cl. CPC C07K 7/08; A01N 37/46; A01P 1/00; A01P 3/00; C07K 14/001

(57) ABSTRACT

Modified synthetic peptides exhibiting improved antimicrobial activity in comparison to wild-type peptide sequences as well as compositions and plant parts which have been treated with the peptides are provided. Methods of using the modified peptides to control microbial infections of plant and animal subjects are also provided.

### CROSS-REFERENCE TO RELATED APPLICATIONS

The present application claims priority to and the benefit of U.S. Provisional Application No. 63/364,077, filed May 3, 2022, the contents of which are herein incorporated by reference in their entirety.

### STATEMENT REGARDING FEDERALLY SPONSORED RESEARCH

This invention was made with government support under National Science Foundation Award Number 2037981 awarded by the National Science Foundation. The government has certain rights in the invention.

### INCORPORATION OF SEQUENCE LISTING

The instant application contains a Sequence Listing which has been submitted electronically in XML format document and which is hereby incorporated by reference in its entirety. Said XML document, created on Apr. 29, 2023, is named 225312-528094_Sequence-listing and is 86,302 bytes in size.

### BACKGROUND

Protection of agriculturally important crops from pathogenic microbes (e.g., fungi, including mold, yeast and dimorphic fungi, or oomycetes) is crucial in improving crop yields.

Fungal infections are a particular problem in damp climates and can become a major concern during crop storage, where such infections can result in spoilage and contamination of food or feed products with fungal toxins. Unfortunately, modern growing methods, harvesting and storage systems can promote plant pathogen infections.

Certain microbes (e.g., fungi, including yeast, or oomycetes) can also be pathogenic to various vertebrates including humans, fish, and the like. Control of plant pathogens is further complicated by the need to simultaneously control multiple microbes of distinct genera. For example, microbes such as Alternaria; Ascochyta; Botrytis; Cercospora; Colletotrichum; Diplodia; Erysiphe; Fusarium; Gaeumannomyces; Helminthosporium; Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Sclerotium; Sclerotinia; Septoria; Thielaviopsis; Uncinula; Venturia; and Verticillium species are all recognized plant pathogens. Consequently, resistant crop plant varieties or antimicrobial agents that control only a limited subset of microbial pathogens can fail to deliver adequate protection under conditions where multiple pathogens are present. It is further anticipated that plant pathogenic microbes can become resistant to existing antimicrobial agents and crop varieties, which can favor the introduction of new microbial control agents with distinct modes of action to combat the resistant microbes.

A group of peptides known as defensins have been shown to inhibit plant pathogens. Defensins have been previously identified as small cysteine-rich peptides of about 45-54 amino acids that constitute an important component of the innate immunity of plants (Sathoff, Phytopathology 109:402 (2019)). Widely distributed in plants, defensins vary greatly in their amino acid composition. However, they all have a compact shape, which is stabilized by either four or five intramolecular disulfide bonds. Plant defensins have been characterized as comprising a conserved γ-core motif comprising a conserved GXCX3-9C (where X is any amino acid) sequence (Lacerda et al., Frontiers in Microbio. 5(116): 1-10 (2014). The three-dimensional structure of the previously characterized γ-core motif consists of two antiparallel 3-sheets, with an interpolated turn region (Ibid.). Antimicrobial activity of certain defensins has been correlated with the presence of positively charged amino acid residues in the γ-core motif (Spelbrink et al., Plant Physiol., 135, 2055-2067 (2004); Sagaram et al., PLoS ONE, 8(12): e82485; (2013)).

Certain nodule specific cysteine rich (NCR) peptides with antimicrobial activity expressed in nodules of Medicago truncatula (Barrel Medic) have been described (WO2010146067). Other NCR peptides from Cicer arietinum (Chickpea) have been described and implicated in the terminal differentiation of endosymbiotic bacteria (Montiel et al., 2016, Molec. Plant Microb. Inter. 29: 210-219). International Patent Application publication WO2020/146360 and US Patent Appl. Pub. 20220061333 describes antimicrobial NCR polypeptides including the CaNCR7, CaNCR13, CaNCR14, and CaNCR15 peptides as well as certain variants thereof. Nodule-specific cysteine-rich (NCR) peptides are normally expressed in the root nodules of leguminous plants, including Cicer arietinum, Medicago truncatula, Galega orientalis, Medicago sativa, Astragalus canadensis, Pisum sativum, Ononis spinosa, Onobrychis viciifolia, and Oxytropis lambertii where they mediate the differentiation of root nodule bacteria into nitrogen-fixing bacteroids thereby maintaining bacterial survival. Wang, Mol. Plant-Microbe Int. 31(2):240-8 (2018), Van de Velde, Science 327:1122-1126 (2010), Kim, PNAS 112:15238-15243 (2015), and Horvath, PNAS 112:15232-15237 (2015).

Certain NCR peptides exhibit antimicrobial properties when applied to free-living bacteria and can mediate bacterial cell death and early nodule senescence. Yang, PNAS 114:6848-6853 (2017) and Wang, PNAS 114:6854-6859 (2017). Antimicrobial NCRs (AMPs) are cationic and have conserved cysteine residues that form intramolecular disulfide bonds. Cysteine substitutions or disulfide bond modifications can influence the antimicrobial activity of certain NCR peptides having just 4 conserved cysteine residues. Haag, J Biol. Chem. 287(14):10791-8 (2012) and Isozumi, Nature Sci. Rep. 11: 9923 (2021).

### SUMMARY

Peptides comprising the amino acid sequence of (a) SEQ ID NO: 1, optionally wherein the C-terminal amino acid residue is amidated; or (b) SEQ ID NO: 5, wherein the peptide does not comprise the amino acid sequence of SEQ ID NO: 9 and optionally wherein the C-terminal amino acid residue is amidated; wherein said peptide comprising SEQ ID NO: 1 or SEQ ID NOs: 5 is 13 to 18, 25, or 30 amino acid residues in length and wherein said peptide contains only the two cysteine residues set forth in SEQ ID NO: 1 or SEQ ID NO: 5 are provided. Also provided are compositions comprising the aforementioned peptides and an agriculturally, pharmaceutically, or veterinary-practicably acceptable carrier, diluent, or excipient. Also provided are medical devices comprising the device and the aforementioned compositions, wherein the device comprises at least one surface that is topically coated and/or impregnated with the composition. Also provided are plant parts that are at least partly coated with the composition.

Methods for preventing or reducing crop damage by a plant pathogenic microbe comprising the step of contacting a plant, a plant seed, or other part of said plant with an effective amount of the aforementioned compositions are also provided. Also provided are methods for treating, preventing, or inhibiting a microbial infection in a subject in need thereof comprising administering to said subject an effective amount of the aforementioned compositions.

Recombinant polynucleotides comprising a polynucleotide encoding a peptide comprising any of the aforementioned peptides are also provided, wherein the polynucleotide encoding the first antimicrobial peptide is operably linked to a polynucleotide comprising a promoter which is heterologous to the polynucleotide encoding the first antimicrobial peptide, optionally wherein any amino acid substitution in the amino acid sequence of the peptide increases or maintains the net positive charge and/or increases or maintains hydrophobicity of the peptide.

Plant nuclear or plastid genomes comprising a polynucleotide encoding a peptide comprising any of the aforementioned peptides, wherein the polynucleotide is heterologous to the nuclear or plastid genome and wherein the polynucleotide is operably linked to an endogenous promoter of the nuclear or plastid genome are provided.

Cells comprising any of the aforementioned recombinant polynucleotides or genomes, wherein the cell is optionally a bacterial, yeast, or plant cell, are provided. Plants comprising any of the aforementioned recombinant polynucleotides or genomes are provided. Plant parts of the aforementioned plants, wherein the plant parts comprise the recombinant polynucleotide or genome, optionally wherein the plant part is a seed, stem, leaf, root, tuber, flower, or fruit, are provided.

Methods for producing plant seed that provides plants resistant to infection by a plant pathogenic microbe that comprises the steps of: (i) selfing or crossing any of the aforementioned plants; and (ii) harvesting seed that comprises the recombinant polynucleotide of the plant from the self or cross, thereby producing plant seed that provide plants resistant to infection by a plant pathogenic microbe, are provided.

### BRIEF DESCRIPTION OF THE FIGURES

FIG. 1 shows an alignment of the CaNCR13 wildtype protein (SEQ ID NO: 38), a truncated CaNCR13 peptide (SEQ ID NO: 41), the extended CaNCR13 peptide variant core sequence (SEQ ID NO: 42), and both conservative and non-conservative amino acid substitutions in the extended CaNCR13 peptide variant core sequence.

FIG. 2 shows an alignment of consensus CaNCR variant peptides (SEQ ID NO: 1, 2, 5, respectively), the CaNCR13_v7 peptide (SEQ ID NO: 9) comprising the corresponding sub-fragment of the wild-type CaNCR13 protein (SEQ ID NO: 38), and CaNCR variant peptides NCR13_v8, NCR13_v9, NCR13_v10, mNCR13_v11, NCR13_v12, and NCR13_v13 (SEQ ID NOs: 10-15, respectively and all in their C-terminally amidated form).

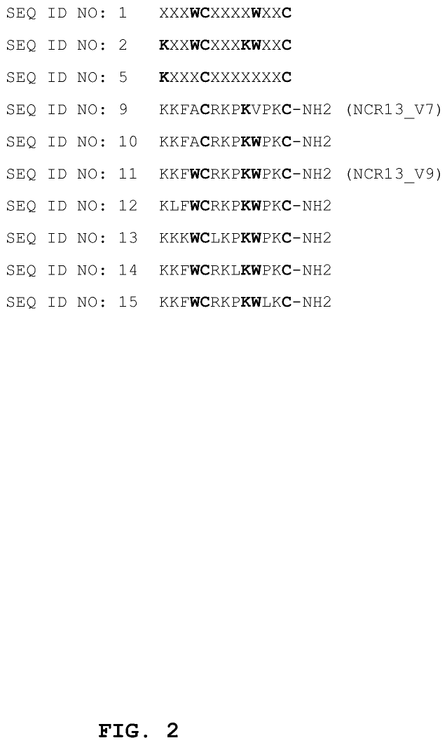

FIG. 3 shows an alignment of the CaNCR7 wild type peptide (SEQ ID NO: 8), the CaNCR14 wild type peptide (SEQ ID NO: 15), and the CaNCR15 wild type peptide (SEQ ID NO: 22) with the full length CaNCR13 wild-type reference peptide of SEQ ID NO: 1 where the CaNCR7, CaNCR14, and CaNCR15 peptides are aligned with SEQ ID NO: 1 at conserved cysteine residues C₁-C₆.

FIG. 4 shows drop-inoculation assays of semi-in planta antifungal activity of NCR13_V7 (SEQ ID NO: 9) and NCR13_V9 (SEQ ID NO: 11) peptides against B. cinerea on the detached leaves of N. benthamiana.

FIG. 5 is a graph that depicts the relative lesion size for the drop-inoculation assays shown in FIG. 4, which tested the semi-in planta antifungal activity of NCR13_V7 (SEQ ID NO: 9) and NCR13_V9 (SEQ ID NO: 11) peptides against B. cinerea on the detached leaves of N. benthamiana.

### DETAILED DESCRIPTION

### Definitions

Amino acid residues in polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

Where a term is provided in the singular, other embodiments described by the plural of that term are also provided.

As used herein, a polynucleotide is said to be “endogenous” to a given cell when it is found in a naturally occurring form and genomic location in the cell.

The phrases “antimicrobial peptide” as used herein refer to peptides which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat).

As used herein, the terms “acidic” or “anionic” are used interchangeably to refer to amino acids such as aspartic acid and glutamic acid.

As used herein, the phrase “CaNCR peptide” refers to CaNCR13 peptide of SEQ ID NO: 38, a CaNCR7 peptide of SEQ ID NO: 37, a CaNCR14 peptide of SEQ ID NO: 39, or a CaNCR15 peptide of SEQ ID NO: 40.

As used herein, the terms “basic” and “cationic” are used interchangeably to refer to amino acids such as arginine, histidine, and lysine.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “combating microbial damage,” “combating or controlling microbial damage” or “controlling microbial damage” as used herein refer to reduction in damage to a crop plant or crop plant product due to infection by a microbial pathogen. More generally, these phrases refer to reduction in the adverse effects caused by the presence of a pathogenic microbe in the crop plant. Adverse effects of microbial (e.g., fungal) growth are understood to include any type of plant tissue damage or necrosis, any type of plant yield reduction, any reduction in the value of the crop plant product, and/or production of undesirable microbial metabolites or microbial growth by-products including but not limited to mycotoxins.

The phrase “defensin peptide” is used herein to refer to a peptide comprising a conserved γ-core motif comprising a conserved GXCX3-9C sequence, where X is any amino acid residue. Defensin peptides include proteins that are antimicrobial, and can bind phospholipids, permeabilize plasma membranes, or bind sphingolipids, or any combination of these properties. A defensin peptide can be naturally occurring or non-naturally occurring (e.g., synthetic and/or chimeric).

As used herein, the terms “edit,” “editing,” “edited” and the like refer to processes or products where insertions, deletions, and/or nucleotide substitutions are introduced into a genome. Such processes include methods of inducing homology directed repair and/or non-homologous end joining of one or more sites in the genome.

The term “endoproteinase” is used herein to refer to a peptidase capable of cleaving a peptide bond between two internal amino acid residues in a peptide sequence. Endoproteinases can also be referred to as “endoproteases” or “endopeptidases.” The proteolytic activity of an endoproteinase, endoprotease, or endopeptidase is thus different that the proteolytic activity of an “exopeptidase” which cleaves peptide bonds of terminal amino acid residues in a peptide.

The phrases “genetically edited plant” or “edited plant” are used herein to refer to a plant comprising one or more nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′UTR, and 3′UTR), translation, or a combination thereof as well as polynucleotides encoding CaNCR peptide variants, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding CaNCR peptide variants, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding CaNCR peptide variants, spacer peptides, or localization can be heterologous to another polynucleotide when the polynucleotides are not operably linked to one another in a naturally occurring cell. Heterologous peptides include peptides that are not found in a cell or organism as the cell or organism occurs in nature. As such, heterologous peptides include peptides that are localized in a subcellular location, extracellular location, or expressed in a tissue that is distinct from the subcellular location, extracellular location, or tissue where the peptide or protein is found in a cell or organism as it occurs in nature. Heterologous polynucleotides include polynucleotides that are not found in a cell or organism as the cell or organism occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and in certain embodiments perform the same biological function in either the same or a different organism.

The phrases “inhibiting growth of a plant pathogenic microbe,” “inhibit microbial growth,” and the like as used herein refers to methods that result in any measurable decrease in microbial growth, where microbial growth includes but is not limited to any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including but not limited to mycotoxins. As used herein, the phrase “inhibition of microbial growth” and the like, unless otherwise specified, can include inhibition in a plant, human or animal.

As used herein, the terms “microbe,” “microbes,” and “microbial” are used to refer to bacteria, fungi (including yeast), and oomycetes.

As used herein, the phrase “CaNCR peptide variant” refers to any peptide with antimicrobial activity comprising a truncated CaNCR peptide having: (i) an N-terminal and/or a C-terminal amino acid residue deletion; and (ii) a selected set of only two of the internal conserved cysteine residues of a CaNCR peptide. Examples of CaNCR peptide variants include peptides set forth in SEQ ID NOs: 1-8 and 42.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5′ untranslated sequence associated with the promoter and the coding sequence is linked such that the resulting translation product is in frame with the translational open reading frame that encodes the protein. Nucleic acid sequences that can be operably linked include sequences that provide gene expression functions (e.g., gene expression elements such as promoters, 5′ untranslated regions, introns, protein coding regions, 3′ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, linker, or other peptide, “operably linked” means that the peptide is connected to the polyprotein sequence(s) of interest such that it provides a function. Functions of a localization peptide include localization of a protein or peptide of interest (e.g., a CaNCR peptide variant or CaNCR peptide variant multimer) to an extracellular space or subcellular compartment. Functions of a spacer peptide include linkage of two peptides of interest (e.g., two CaNCR peptide variants) such that the peptides will be expressed as a single protein (e.g., a CaNCR peptide variant multimer).

The phrases “percent identity” or “sequence identity” as used herein refer to the number of elements (i.e., amino acids or nucleotides) in a sequence that are identical within a defined length of two DNA, RNA or protein segments in an alignment resulting in the maximal number of identical elements, and is calculated by dividing the number of identical elements by the total number of elements in the defined length of the aligned segments and multiplying by 100.

The terms “susceptible microbe (or microbes),” “susceptible microbial infection,” and the like refer to microbes that infect plants, or human or animal patients or subjects, or microbial infections thereof, that are subjection to inhibition of microbial growth by the CaNCR peptide variants or CaNCR peptide variant multimers disclosed herein.

The phrase “transgenic” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Truncated variants of antimicrobial nodule specific cysteine rich peptides referred to as CaNCR peptide variants comprising N-terminal and/or C-terminal deletions of a CaNCR peptide and containing just 2 of the conserved cysteines of a CaNCR peptide are provided herein. The antimicrobial CaNCR peptide variants can be applied directly to a plant, applied to a plant in the form of microorganisms that produce the CaNCR peptide variant, or the plants can be transformed or genetically edited to produce the CaNCR peptide variant. In certain embodiments, antimicrobial CaNCR peptide variants provided herein can be provided in compositions or used under conditions where the levels of sodium and/or calcium salts exceed levels where a corresponding CaNCR peptide or CaNCR sub-peptide exhibits decreased antifungal activity (e.g., sodium salt at a concentration of at least 100 mM and/or a calcium salt at a concentration of at least 2 mM). The present disclosure also relates to recombinant or edited polynucleotides, microorganisms and plants transformed with the recombinant nucleic acids, plants comprising genetically edited nuclear or plastid genomes encoding the CaNCR peptide variants or CaNCR peptide variant multimers and compositions comprising the CaNCR peptide variants or CaNCR peptide variant multimers useful in controlling pathogenic microbes including, but not limited to, plant, animal, and human pathogenic microbes.

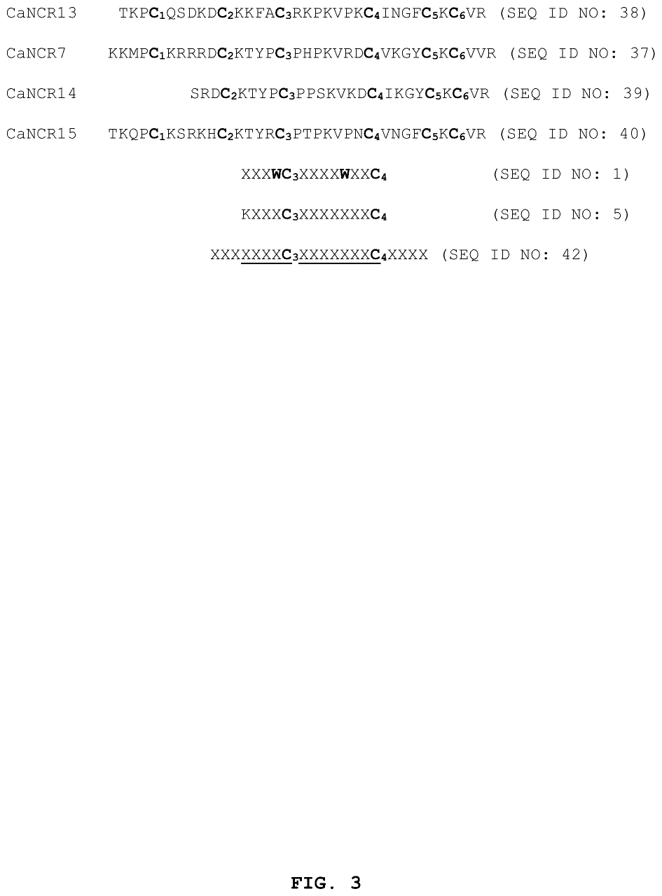

In certain embodiments, a structural feature of CaNCR variant peptides is the presence of just 2 of the 5 or 6 conserved cysteine residues set forth in the CaNCR peptides CaNCR13 (SEQ ID NO: 38), CaNCR7 (SEQ ID NO: 37), CaNCR14 (SEQ ID NO: 39), and CaNCR15 (SEQ ID NO: 40). Such conserved cysteine residues are depicted in FIG. 3. Conserved cysteines comprise C₁, C₂, C₃, C₄, C₅, and C₆ in the CaNCR peptides of CaNCR13 (SEQ ID NO: 38), CaNCR7 (SEQ ID NO: 37), and CaNCR15 (SEQ ID NO: 40), where the cysteine residue closest to the amino terminus is C₁ and the cysteine residue closest to the carboxy terminus is C₆. CaNCR14 (SEQ ID NO: 39) lacks the N-terminal C₁ conserved cysteine. In certain embodiments, CaNCR peptide variants will contain only the two cysteine residues corresponding to the conserved C₃ and C₄ residues in the CaNCR peptides CaNCR13 (SEQ ID NO: 38), CaNCR7 (SEQ ID NO: 37), CaNCR14 (SEQ ID NO: 39), and CaNCR15 (SEQ ID NO: 40). Two cysteine residues corresponding to the conserved C₃ and C₄ in the CaNCR peptides are present in the CaNCR peptide variant consensus sequences of SEQ ID NOs 1-8 and CaNCR variant peptides of SEQ ID NOs: 10-15, 17-23, and 25-35. The two cysteine residues corresponding to the conserved C₃ and C₄ residues in the CaNCR peptides are present in the CaNCR peptide variant consensus sequences of SEQ ID NO: 9 when the C₂ residue is substituted with an amino acid residue other than cysteine (e.g., as in Table 9). In certain embodiments, the cysteine residue in the CaNCR variant peptide corresponding to the C₄ residue is the C-terminal residue of the CaNCR variant peptide and is optionally amidated. In certain embodiments, the C-terminal residue of the CaNCR variant peptide comprises one or more additional amino residues other than cysteine located C-terminal to the C₄ cysteine residue in the CaNCR variant peptide, where the C-terminal amino acid residue is optionally amidated. The CaNCR peptide variant consensus sequence of SEQ ID NO: 42 comprises one or more additional amino residues other than cysteine located C-terminal to the C₄ cysteine. In certain embodiments, the conserved C₃ and C₄ residues will have a disulfide bridge in the CaNCR variant peptide.

CaNCR peptide variants provided herein can comprise a deletion of 4, 5, 6, 7, 8, 9, or 10 amino acid residues from the N-terminus and/or the C-terminus of a CaNCR peptide of SEQ ID NOs: 37, 38, 39, or 40, a conservatively substituted variant thereof, and/or a non-conservatively substituted variant thereof. In certain embodiments, a CaNCR13 peptide variant provided herein can comprise a deletion of at least 4, 5, 6, 7, 8, 9, or 10 amino acid residues from the N-terminus and/or a deletion of at least 4, 5, 6, 7, 8, or 9 amino acid residues from the C-terminus of a CaNCR13 peptide of SEQ ID NO: 38, a conservatively substituted variant thereof, and/or a non-conservatively substituted variant thereof. In certain embodiments, a CaNCR7 peptide variant provided herein can comprise a deletion of at least 4, 5, 6, 7, 8, 9, or 10 amino acid residues from the N-terminus and/or a deletion of 5, 6, 7, 8, 9, or 10 amino acid residues from the C-terminus of a CaNCR7 peptide of SEQ ID NO: 37, a conservatively substituted variant thereof, and/or a non-conservatively substituted variant thereof. In certain embodiments, a CaNCR14 peptide variant provided herein can comprise a deletion of at least 4 amino acid residues from the N-terminus and/or a deletion of 5, 6, 7, 8, or 9 amino acid residues from the C-terminus of a CaNCR14 peptide of SEQ ID NO: 39, a conservatively substituted variant thereof, and/or a non-conservatively substituted variant thereof. In certain embodiments, a CaNCR13 peptide variant provided herein can comprise a deletion of at least 5, 6, 7, 8, 9, 10, or amino acid residues from the N-terminus and/or a deletion of 5, 6, 7, 8, or 9 amino acid residues from the C-terminus of a CaNCR13 peptide of SEQ ID NO: 40, a conservatively substituted variant thereof, and/or a non-conservatively substituted variant thereof. In instances wherein the N- and/or C-terminal amino acid deletion(s) are insufficient to remove cysteine residues corresponding to C₁, C₂, C₅, and/or C₆ in the CaNCR peptides, the remaining cysteine residues corresponding to C₁, C₂, C₅, and/or C₆ are substituted with non-cysteine residues. In certain embodiments, one or more of the remaining cysteine residues corresponding to the C₁, C₂, C₅, and/or C₆ cysteine residue can be substituted with another amino acid residue including a glycine, serine, threonine, asparagine, or glutamine residue. In certain embodiments, aforementioned N-terminal deletions can further comprise a substitution the remaining N-terminal residue of the N-terminally deleted CaNCR peptide with an alanine residue.

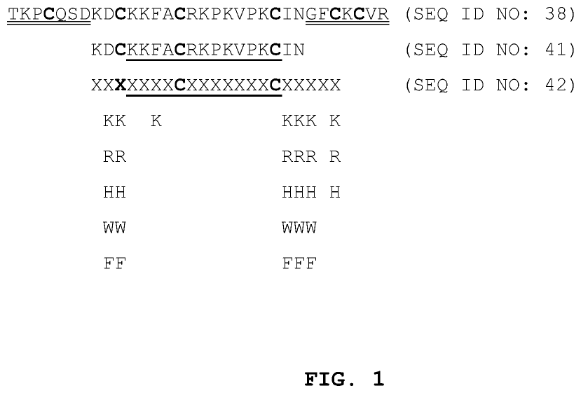

Tables 1-9 provide a non-limiting sample of CaNCR peptide variants provided herein, where the C-terminal cysteine residue corresponding to the conserved C₄ cysteine of a CaNCR peptide or other C-terminal amino acid residue is optionally amidated. In certain embodiments, CaNCR variant peptides of Tables 1-9 can comprise any combination of the residues set forth under the consensus sequence (e.g., amino acids Xaa or X are each independently selected from amino acids listed in the column beneath each Xaa or X amino acids Xaa or X in Tables 1-9) and further comprise one, two, three, or more additional amino acids at their N-terminus and/or C-terminus, wherein the N-terminal amino acid is optionally an alanine residue and/or wherein the C-terminal residue is optionally amidated. In certain aforementioned embodiments, the CaNCR variant peptides can comprise the consensus sequence of SEQ ID NO: 42 set forth in Table 9. In certain aforementioned embodiments, the CaNCR variant peptides comprising the consensus sequence of SEQ ID NO: 42 can comprise any one or more of the amino acid substitutions set forth in FIG. 1 or in Tables 1-8. In certain embodiments, the CaNCR variant peptides comprising the consensus sequence of SEQ ID NO: 42 can comprise N-terminal amino acid residues 1 to 2 which are the corresponding amino acid residues of SEQ ID NOs: 37, 38, 39, or 40 as aligned in FIG. 3. In certain embodiments, the CaNCR variant peptides comprising the consensus sequence of SEQ ID NO: 42 can comprise amino acids located C-terminal to the most C-terminal cysteine, which are the corresponding amino acid residues of SEQ ID NOs: 37, 38, 39, or 40 as aligned in FIG. 3.

TABLE 1
CaNCR peptide variant consensus 1
SEQ ID NO: 1 CONSENSUS 1 (XXXWCXXXXWXXC)

| | | | | | | | | | | | | |
|---|---|---|---|---|---|---|---|---|---|---|---|---|
| Xaa1 | Xaa2 | Xaa3 | W | C | Xaa6 | Xaa7 | Xaa8 | Xaa9 | W | Xaa11 | Xaa12 | C |

| | | | | | | | | |
|---|---|---|---|---|---|---|---|---|
| K | K | F | R | K | P | K | P | K |
| R | R | Y | K | R | S | R | K | W |
| H | H | W | P | P | L | | R | R |
| | T | K | H | H | | | H | H |
| | L | | L | T | | | L | |

TABLE 2
CaNCR peptide variant subconsensus 1A
SEQ ID NO: 2 SUBCONSENSUS 1A (KXXWCXXXKWXXC) Species Set 1

| | | | | | | | | | | | | |
|---|---|---|---|---|---|---|---|---|---|---|---|---|
| K | Xaa2 | Xaa3 | W | C | Xaa6 | Xaa7 | Xaa8 | K | W | Xaa11 | Xaa12 | C |

| | | | | | | |
|---|---|---|---|---|---|---|
| K | F | R | K | P | P | K |
| R | Y | K | R | S | K | W |
| H | W | P | P | L | R | R |
| T | K | H | H | | L | |
| L | | L | T | | | |

TABLE 3
CaNCR peptide variant subconsensus 1B.
SEQ ID NO: 3 SUBCONSENSUS 1B (KXXWCXXXKWXXC) Species Set 2

| | | | | | | | | | | | | |
|---|---|---|---|---|---|---|---|---|---|---|---|---|
| K | Xaa2 | Xaa3 | W | C | Xaa6 | Xaa7 | Xaa8 | K | W | Xaa11 | Xaa12 | C |

| | | | | | | |
|---|---|---|---|---|---|---|
| K | F | R | K | P | P | K |
| R | W | K | R | S | K | W |
| T | | P | P | | R | R |
| | | | H | | H | H |
| | | | T | | | |

TABLE 4
CaNCR peptide variant subconsensus 1C.
SEQ ID NO: 4 SUBCONSENSUS 1C (KXXWCXXXKWXXC) Species Set 3

| | | | | | | | | | | | | |
|---|---|---|---|---|---|---|---|---|---|---|---|---|
| K | Xaa2 | Xaa3 | W | C | Xaa6 | Xaa7 | Xaa8 | K | W | Xaa11 | Xaa12 | C |

| | | | | | | |
|---|---|---|---|---|---|---|
| K | F | R | K | P | P | K |
| R | W | K | R | S | K | W |
| | | P | P | | R | R |

TABLE 5
CaNCR peptide variant consensus 2.
SEQ ID NO: 5 CONSENSUS 2 (XXXXCXXXXXXXC)

| | | | | | | | | | | | | |
|---|---|---|---|---|---|---|---|---|---|---|---|---|
| Xaa1 | Xaa2 | Xaa3 | Xaa4 | C | Xaa6 | Xaa7 | Xaa8 | Xaa9 | Xaa10 | Xaa11 | Xaa12 | C |

| | | | | | | | | | | |
|---|---|---|---|---|---|---|---|---|---|---|
| K | K | F | A | R | K | P | K | W | P | K |
| R | R | Y | W | K | R | S | R | V | K | W |
| H | H | W | P | P | P | L | | F | R | R |
| | T | K | F | H | H | | | Y | H | H |
| | L | | Y | L | T | | | | L | |

TABLE 6
CaNCR peptide variant sub-consensus 2A.
SEQ ID NO: 6 SUB-CONSENSUS 2A (KXXXCXXXXXXXC)

| | | | | | | | | | | | | |
|---|---|---|---|---|---|---|---|---|---|---|---|---|
| Xaa1 | Xaa2 | Xaa3 | Xaa4 | C | Xaa6 | Xaa7 | Xaa8 | K | Xaa10 | Xaa11 | Xaa12 | C |

| | | | | | | | | | |
|---|---|---|---|---|---|---|---|---|---|
| K | K | F | A | R | K | P | W | P | K |
| R | R | Y | W | K | R | S | V | K | W |
| H | H | W | P | P | P | L | | R | R |
| | T | K | F | H | H | | | L | |
| | L | | | L | T | | | | |

TABLE 7
CaNCR peptide variant sub-consensus 2B.
SEQ ID NO: 7 SUB-CONSENSUS 2B (KXXXCXXXXXXXC)

| | | | | | | | | | | | | |
|---|---|---|---|---|---|---|---|---|---|---|---|---|
| Xaa1 | Xaa2 | Xaa3 | Xaa4 | C | Xaa6 | Xaa7 | Xaa8 | K | Xaa10 | Xaa11 | Xaa12 | C |

| | | | | | | | | | |
|---|---|---|---|---|---|---|---|---|---|
| K | K | F | A | R | K | P | W | P | K |
| R | R | Y | W | K | R | S | V | K | W |
| H | H | W | P | P | P | | F | R | |
| | T | K | F | | H | | | | |
| | | | | | T | | | | |

TABLE 8
CaNCR peptide variant sub-consensus 2C.
SEQ ID NO: 8 SUB-CONSENSUS 2C (KXXXCXXXXXXXC)

| | | | | | | | | | | | | |
|---|---|---|---|---|---|---|---|---|---|---|---|---|
| K | Xaa2 | Xaa3 | Xaa4 | C | Xaa6 | Xaa7 | Xaa8 | K | Xaa10 | Xaa11 | Xaa12 | C |

| | | | | | | | | |
|---|---|---|---|---|---|---|---|---|
| K | F | A | R | K | P | W | P | K |
| R | Y | W | K | R | S | V | K | W |
| | W | P | P | P | | F | R | |
| | | F | | | | | | |

TABLE 9
Extended CaNCR peptide variant Consensus (SEQ ID NO: 42)

| | | | | | | | | | | | | | | | | | | | |
|---|---|---|---|---|---|---|---|---|---|---|---|---|---|---|---|---|---|---|---|
| X | X | X | X | X | X | X | C | X | X | X | X | X | X | X | C | X | X | X | X |

| | | | | | | | | | | | | | | | | | |
|---|---|---|---|---|---|---|---|---|---|---|---|---|---|---|---|---|---|
| K | K | K | K | K | F | W | R | K | P | K | W | P | K | I | N | G | F |
| R | R | H | R | R | Y | F | K | R | S | R | F | K | W | V | K | A | Y |
| S | H | R | H | H | W | Y | P | P | L | H | Y | R | R | A | R | K | W |
| H | W | W | | T | H | | L | T | | | | L | | K | H | F | K |
| A | F | F | | L | R | | H | H | | | | | | F | F | Y | |
| | Y | Y | | | K | | | | | | | | | Y | Y | | |
| | | | | | | | | | | | | | | L | | | |

TABLE 10
Summary of Peptide Sequences

| | SEQ | | |
|---|---|---|---|
| | ID | | |
| Peptide Name | NO | SEQUENCE | COMMENT |
| CONSENSUS | 1 | XXXWCXXXXWXXC | Where X |
| 1 | | | residues are set |
| | | | forth in Table 1 |
| SUB | 2 | KXXWCXXXKWXXC | Where X |
| CONSENSUS | | | residues are set |
| 1A | | | forth in Table 2 |
| SUB | 3 | KXXWCXXXKWXXC | Where X |
| CONSENSUS | | | residues are set |
| 1B | | | forth in Table 3 |
| SUB | 4 | KXXWCXXXKWXXC | Where X |
| CONSENSUS | | | residues are set |
| 1C | | | forth in Table 4 |
| CONSENSUS | 5 | XXXXCXXXXXXXC | Where X |
| 2 | | | residues are set |
| | | | forth in Table 5 |
| SUB | 6 | XXXXCXXXKXXXC | Where X |
| CONSENSUS | | | residues are set |
| 2A | | | forth in Table 6 |
| SUB | 7 | KXXXCXXXKXXXC | Where X |
| CONSENSUS | | | residues are set |
| 2B | | | forth in Table 7 |
| SUB | 8 | KXXXCXXXKXXXC | Where X |
| CONSENSUS | | | residues are set |
| 2C | | | forth in Table 8 |
| NCR13_v7 | 9 | KKFACRKPKVPKC-NH₂ | WILD TYPE |
| | | | CaNCR13 with |
| | | | amidation |
| NCR13_v8 | 10 | KKFACRKPKWPKC-NH₂ | CONSENSUS |
| | | | 2 species |
| NCR13_v9 | 11 | KKFWCRKPKWPKC-NH₂ | CONSENSUS |
| | | | 1 species |
| NCR13_v10 | 12 | KLFWCRKPKWPKC-NH₂ | CONSENSUS |
| | | | 1 species |
| NCR13_v11 | 13 | KKKWCLKPKWPKC-NH₂ | CONSENSUS |
| | | | 1 species |
| NCR13_v12 | 14 | KKFWCRKLKWPKC-NH₂ | CONSENSUS |
| | | | 1 species |
| NCR13_v13 | 15 | KKFWCRKPKWLKC-NH₂ | CONSENSUS |
| | | | 1 species |
| NCR14_v5 | 16 | KTYPCPPSKVKDC-NH₂ | WILD TYPE |
| | | | CaNCR14 with |
| | | | amidation |
| NCR14_v6 | 17 | KTYPCPPSKVKWC-NH₂ | CONSENSUS |
| | | | 2 species |
| NCR14_v7 | 18 | KKYPCPPSKWKWC-NH₂ | CONSENSUS |
| | | | 2 species |
| NCR14_v9 | 19 | KTWPCPPSKWKKC-NH₂ | CONSENSUS |
| | | | 2 species |
| NCR14_v10 | 20 | KKWPCPPSKWKKC-NH₂ | CONSENSUS |
| | | | 2 species |
| NCR14_v11 | 21 | KTYWCPPSKWKDC-NH₂ | CONSENSUS |
| | | | 1 species |
| NCR14_v12 | 22 | KKYWCPPSKWKKC-NH₂ | CONSENSUS |
| | | | 1 species |
| NCR14_v13 | 23 | KKFWCPPSKWKKC-NH₂ | CONSENSUS |
| | | | 1 species |
| NCR15_v5 | 24 | KTYRCPTPKVPNC-NH₂ | WILD TYPE |
| | | | CaNCR15 with |
| | | | amidation |
| NCR15_v6 | 25 | KTYRCPTPKWPKC-NH₂ | CONSENSUS |
| | | | 2 |
| NCR15_v7 | 26 | KKWRCPTPKWPKC-NH₂ | CONSENSUS |
| | | | 2 |
| NCR15_v8 | 27 | KTWRCPPPKWPKC-NH₂ | CONSENSUS |
| | | | 2 |
| NCR15_v9 | 28 | KKWRCPPPKWPKC-NH₂ | CONSENSUS |
| | | | 2 |
| NCR15_v10 | 29 | KKYWCPTPKWPKC-NH₂ | CONSENSUS |
| | | | 1 |
| NCR15_v11 | 30 | KKFWCPTPKWPKC-NH₂ | CONSENSUS |
| | | | 1 |
| NCR7_v5 | 31 | KKYPCPHPKWRKC-NH₂ | CONSENSUS |
| | | | 2 |
| NCR7_v6 | 32 | KKWPCPHPKWRKC-NH₂ | CONSENSUS |
| | | | 2 |
| NCR7_v7 | 33 | KKWPCPKPKWRKC-NH₂ | CONSENSUS |
| | | | 2 |
| NCR7_v8 | 34 | KKYWCPHPKWRKC-NH₂ | CONSENSUS |
| | | | 1 |
| NCR7_v9 | 35 | KKFWCPHPKWRKC-NH₂ | CONSENSUS |
| | | | 1 |
| NCR7_v10 | 36 | KTYPCPHPKVRDC-NH₂ | Wild-type |
| | | | CaNCR7 with |
| | | | amidation |
| NCR7 | 37 | KKMPCKRRRDCKTYPCPHPKVRDCVKGYCK | Wild-type |
| | | CVVR | mature |
| | | | CaNCR7 |
| | | | peptide |
| NCR13 | 38 | TKPCQSDKDCKKFACRKPKVPKCINGFCKCVR | Wild-type |
| | | | mature |
| | | | CaNCR13 |
| | | | peptide |
| NCR14 | 39 | SRDCKTYPCPPSKVKDCIKGYCKCVR | Wild-type |
| | | | mature |
| | | | CaNCR14 |
| | | | peptide |
| NCR15 | 40 | TKQPCKSRKHCKTYRCPTPKVPNCVNGFCKC | Wild-type |
| | | VR | mature |
| | | | CaNCR15 |
| | | | peptide |
| NCR13 | 41 | KDCKKFACRKPKVPKCIN | Wild-type |
| truncated | | | CaNCR13 |
| wild-type | | | peptide |
| | | | fragment |
| NCR 13 | 42 | XXXXXXXCXXXXXXXCXXXX | Where X |
| Extended | | | residues are set |
| Consensus | | | forth in Table 9 |
| NCR13_AlaV | 43 | TKPCAAAAACKKFACRKPKVPKCINGFCKCV | |
| 1 | | R | |
| NCR13_AlaV | 44 | TKPCQSDKDCAAAACRKPKVPKCINGFCKCV | |
| 2 | | R | |
| NCR13_AlaV | 45 | TKPCQSDKDCKKFACAAAAVPKCINGFCKCV | |
| 3 | | R | |
| NCR13_AlaV | 46 | TKPCQSDKDCKKFACRKPKAAACINGFCKCV | |
| 4 | | R | |
| NCR13_AlaV | 47 | TKPCQSDKDCKKFACRKPKVPKCAAAACKCV | |
| 5 | | R | |
| NCR13 core | 48 | KKFACRKPKV | |
| Yeast Protease | 49 | KREAEA | |
| Cleavage site | | | |

CaNCR peptide variants provided herein can comprise a peptide having conservative and/or non-conservative amino acid substitutions, and internal deletions of one or more amino acid residues, all relative to the corresponding CaNCR peptide. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (anionic; negatively charged) amino acids such as aspartic acid and glutamic acid; (2) basic (cationic; positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within CaNCR peptide variant sequences can be made by substituting one amino acid within one of these groups with another amino acid within the same group. Non-conservative amino acid changes within CaNCR peptide variant sequences can be made by substituting one amino acid in one of these groups with another amino acid in a different group. Examples of CaNCR peptide variants provided herein include SEQ ID NOs: 1-15, 17-23, 25-35, and 42 as well as peptides comprising conservative and non-conservative amino acid substitutions in SEQ ID NOs: 10-15, 17-23, and 25-35.

In certain embodiments, a structural feature of the CaNCR peptide variants is a net positive charge at neutral pH. In certain embodiments, the CaNCR peptide variants will have a net positive charge at neutral pH of at least +5, +6, +7, +8, +9, or +10. In certain embodiments, the CaNCR peptide variants will have a net positive charge at neutral pH of at least +4, +5, +6, +7, +8, +9, or +10 to +12, +13, +14, or +15. In certain embodiments, such net positive charges in CaNCR peptide variants can be achieved by methods that include: (i) maintaining cationic (basic) amino acid residues found in CaNCR peptide variants including SEQ ID NOs: 10 to 15, 17 to 23, and 25 to 35 or substituting such residues with another cationic amino acid residue; (ii) substituting anionic or polar amino acid residues found in CaNCR peptide variants including SEQ ID NO: with a basic amino acid residue; or a combination of (i) and (ii). In certain embodiments, such net positive charges in CaNCR peptide variants can be achieved by preferentially selecting or substituting a cationic amino acid residue at variable positions in the CaNCR peptide variant that correspond to a variable position of SEQ ID NOs: 1 to 8 or 42. For example, a cationic amino acid can be preferentially selected or substituted for any of the variable amino acids of SEQ ID NO: 1 to 8 or 42 as set forth in Tables 1 to 9.

In certain embodiments, a structural feature of the CaNCR peptide variants is a significant percentage of hydrophobic amino acid residues. In certain embodiments, the CaNCR peptide variants will comprise at least about 25%, 26%, 28% 30%, 32%, 34%, 36%, 37%, or 38% hydrophobic amino acid residues. In certain embodiments, the CaNCR peptide variants will comprise at least about 25%, 26%, 28% 30%, 32%, 34%, or 36% to 37%, 38%, 40%, 42%, or 45% hydrophobic amino acid residues. In certain embodiments, such percentages of hydrophobic amino acids in CaNCR peptide variants can be achieved by methods that include: (i) maintaining hydrophobic amino acid residues found in CaNCR peptide variants including SEQ ID NOs: 10 to 15, 17 to 23, and 25 to 35 or substituting such residues with another hydrophobic amino acid residue or neutral polar amino acid residue (e.g., with a hydrophobic amino acid substitution corresponding to a hydrophobic amino substitution in as shown in Tables 1-9 and/or FIG. 1); (ii) substituting polar amino acid residues found in CaNCR peptide variants including SEQ ID NOs: 10 to 15, 17 to 23, and 25 to 35 with a hydrophobic amino acid residue; (iii) substituting neutral polar amino acids for hydrophobic amino acids; or a combination of (i), (ii), and (iii). Examples of such substitutions of amino acid residues in certain CaNCR peptide variants to increase hydrophobicity include those corresponding to substitutions of certain residues set forth in the CaNCR13 peptide of SEQ ID NO: 38 shown in FIG. 1. In certain embodiments, such percentages of hydrophobic amino acids in CaNCR peptide variants can be achieved by preferentially selecting or substituting a hydrophobic amino acid residue at variable positions in the CaNCR peptide variant that correspond to a variable position of SEQ ID NOs: 1-8 or 42 as set forth in Tables 1 to 9.

In any of the aforementioned embodiments, the CaNCR peptide variant(s) can comprise an amino acid sequence that is at least 60%, 70%, 75%, 80%, 85%, 90%, 95%, 98%, 99%, or 100% identical to SEQ ID NOs: 10 to 15, 17 to 23, and 25 to 35, provided that the CaNCR peptide variant does not comprise the corresponding amino acid sequence of SEQ ID NOs: 37, 38, 39, or 40. In certain aforementioned embodiments, the CaNCR peptide variant(s) comprising an amino acid sequence that is at least 60%, 70%, 75%, 80%, 85%, 90%, 95%, 98%, or 99% identical to SEQ ID NOs: 10 to 15, 17 to 23, and 25 to 35 can comprise a peptide wherein one or more of the acidic, basic, neutral polar, and/or neutral non-polar amino acid residues of SEQ ID NOs: 10 to 15, 17 to 23, and 25 to 35 are conservatively substituted with other acidic, basic, neutral polar, and/or neutral non-polar amino acid residues, respectively. In certain aforementioned embodiments, the CaNCR peptide variant(s) comprising an amino acid sequence that is at least 60%, 70%, 75%, 80%, 85%, 90%, 95%, 98%, or 99% identical to SEQ ID NOs: 10 to 15, 17 to 23, and 25 to 35 can comprise a peptide wherein one or more of the acidic, basic, neutral polar, and/or neutral non-polar amino acid residues of SEQ ID NOs: 10 to 15, 17 to 23, and 25 to 35 are non-conservatively substituted with other acidic, basic, neutral polar, and/or neutral non-polar amino acid residues. Biologically functional equivalents of CaNCR peptide variants can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the CaNCR peptide variants. In certain embodiments, an amino acid residue in SEQ ID NOs: 1-10 to 15, 17 to 23, and 25 to 35 is substituted with a corresponding conserved or non-conserved amino acid residue located at the same position in the CaNCR13, CaNCR7, CaNCR14 or CaNCR15 peptides of SEQ ID NOs: 38, 37, 39, or 40, respectively, as per the alignment of FIG. 3. In one non-limiting example, the amino acid residue immediately N-terminal to conserved cysteine C₃ can be any of an alanine, proline, or arginine and/or the amino acid residue immediately C-terminal to conserved cysteine C₃ can be proline or arginine.

A CaNCR peptide variant provided herein can be operably linked to another or the same CaNCR peptide variant, defensin, or antimicrobial peptide via a linker peptide sequence to obtain a CaNCR peptide variant multimer that is susceptible to cleavage by an endoproteinase, including a plant endoproteinase. In certain embodiments, the resultant CaNCR peptide variant multimer can be expressed in a cell such that the endoproteinase cleaves the CaNCR peptide variant multimer to provide the CaNCR peptide variant(s) and/or CaNCR peptide variant and defensin or another antimicrobial peptide. Such CaNCR peptide variant multimers can be provided in a cellular compartment (e.g., cytoplasm, mitochondria, plastid, vacuole, or endoplasmic reticulum) or extracellular space (i.e., to the apoplast) having an endoproteinase that cleaves the linker peptide. Cleavable linker peptides are disclosed in WO2014078900, Vasivarama and Kirti, 2013a, François et al., Vasivarama and Kirti, 2013b, US Patent Appl. Pub. 20190194268, and US Patent Appl. Pub. 20220061333, which are each incorporated herein by reference in their entireties, can be used in the CaNCR peptide variant multimers provided herein.

In certain embodiments, the permeability of a microbial plasma membrane treated with the CaNCR peptide variants is increased in comparison to permeability of a microbial plasma membrane treated with a CaNCR peptide ((e.g., a peptide comprising SEQ ID NOs: 9, 16, 24, or 36). Membrane permeability can be measured by a variety of techniques that include dye uptake. Convenient dye uptake assays that can be used to assess changes in in membrane permeability include assays for uptake of Hoechst 33342 (H0342), rhodamine 123, SYTOX™ Green, and the like. These dyes enter into microbial cells only if their plasma membrane has been permeabilized by a defensin or another membrane-permeabilizing agent. Without seeking to be limited by theory, in certain embodiments it is believed that the CaNCR peptide variant can provide improved microbial inhibition by increasing the permeability of treated microbial membranes in comparison to microbial membranes treated with a CaNCR peptide.

In certain embodiments, the CaNCR peptide variant can exhibit binding to a phospholipid. In certain embodiments, CaNCR peptide variants provided herein can exhibit lower IC50 and/or MIC50 values against one or more microbial pathogens, improved binding to phospholipids, or any combination thereof in comparison to a reference peptide containing a CaNCR peptide. In certain embodiments, CaNCR peptide variant can be optimized for lower IC50 and/or MIC50 values against one or more microbial pathogens by selecting for CaNCR peptide variant that provide for improved phospholipid binding in comparison to a reference protein containing just one of the defensin peptides. Suitable assays for determining improved phospholipid include protein-lipid overlay assays (e.g., Dowler et al., 2002), surface plasmon resonance assays (e.g., Baron and Pauron, 2014), biotin capture lipid affinity assays (e.g., Davidson et al., 2006), titration calorimetry assays (e.g., Miller and Cistola, 1993), and the like.

Expression cassettes that provide for expression of the CaNCR peptide variant in monocotyledonous plants, dicotyledonous plants, or both can be constructed. Such CaNCR peptide variant or protein expression cassette construction can be effected either in a plant expression vector or in the genome of a plant. Expression cassettes are DNA constructs wherein various promoter, coding, and polyadenylation sequences are operably linked. In general, expression cassettes typically comprise a promoter that is operably linked to a sequence of interest, which is operably linked to a polyadenylation or terminator region. In certain instances including, but not limited to, the expression of recombinant or edited polynucleotides in monocot plants, it can also be useful to include an intron sequence. When an intron sequence is included, it is typically placed in the 5′ untranslated leader region of the recombinant or edited polynucleotide. In certain instances, it can also be useful to incorporate specific 5′ untranslated sequences in a recombinant or edited polynucleotide to enhance transcript stability or to promote efficient translation of the transcript.

In certain embodiments, a plant comprising a recombinant or edited polynucleotide encoding a CaNCR peptide variant can be obtained by using techniques that provide for site specific insertion of heterologous DNA into the genome of a plant (e.g., by editing). In certain embodiments, the DNA fragment comprising a CaNCR peptide variant is site specifically integrated into the genome to a plant cell, tissue, part, or whole plant to create a sequence within that genome that encodes a CaNCR peptide variant. In one non-limiting example, a heterologous DNA encoding a CaNCR peptide variant can be inserted into an endogenous genomic region encoding an endogenous peptide at the same time that a heterologous promoter, promoter element, and/or localization peptide is inserted into the genomic region. Examples of methods for inserting foreign DNA at specific sites in the plant genome with site-specific nucleases such as meganucleases or zinc-finger nucleases are at least disclosed in Voytas, 2013. Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Murovec et al., 2017; Kumar and Jain, 2015; and in US Patent Appl. Pub. No. 20150082478, which is specifically incorporated herein by reference in its entirety. Examples of additional methods for editing plant genomes through use of Cpf1 or Csm1 nucleases are disclosed in US Patent Application Publication 20180148735, which is incorporated herein by reference in its entirety.

Transgenic plants can also be obtained by linking a gene of interest (in this case a CaNCR peptide variant-encoding polynucleotide sequence) to a scoreable marker gene, introducing the linked polynucleotides into a plant cell by any of the methods described above, and regenerating the transgenic plants from transformed plant cells that test positive for expression of the scoreable marker gene. The scoreable marker gene can be a gene encoding a beta-glucuronidase protein, a green fluorescent protein, a yellow fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein, or a chloramphenicol acetyl transferase protein.

CaNCR peptide variants can be synthesized de novo from a CaNCR peptide variant sequence disclosed herein or expressed from a nucleotide sequence encoding a CaNCR peptide variant. The sequence of the peptide or protein-encoding nucleotide sequence can be deduced from the NCR peptide sequence by reference to the genetic code. Computer programs such as “BackTranslate” (GCG™ Package, Acclerys, Inc. San Diego, CA) can be used to convert a peptide sequence to the corresponding nucleotide sequence that encodes the peptide.

Expression of CaNCR peptide variants in yeast and filamentous fungi to produce the CaNCR peptide variant is specifically contemplated herein. The construction of expression vectors for production of heterologous proteins in various yeast genera is well established. In general, such expression vectors typically comprise a promoter that is operably linked to a sequence of interest that is operably linked to a polyadenylation or terminator region. Examples of yeast genera that have been used to express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985) Biotechnol Genet Eng Rev. 3:377-416. Expression vectors and transformation systems useful for yeasts other than Saccharomyces are described in Reiser et al (1990) Adv Biochem Eng Biotechnol.; 43:75-102. Other examples of fungal systems which can be adapted for use in expressing CaNCR peptide variants include filamentous fungal systems such as Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes and Trichoderma systems (e.g., U.S. Pat. Nos. 11,046,736 and 11,180,767, each incorporated herein by reference in its entirety). Other systems that can be adapted for use in expressing CaNCR peptide variants include Chrysosporium lucknowense systems (e.g., U.S. Pat. Nos. 8,871,493 and 9,175,296, each incorporated herein by reference in its entirety).

Expression of CaNCR peptide variants in bacterial cells including Escherichia sp. (e.g., E. coli) to produce a CaNCR peptide variant is also specifically contemplated herein. Systems for expressing proteins that comprise disulfide bonds can be adapted for expression of the CaNCR peptide variants in E. coli include those disclosed in US Patent Application US20200172915, which is incorporated herein by reference in its entirety, and in Berkmen, M. Protein Expr Purif. 82(1):240-51 (2012). Other systems useful for expression of proteins which comprise disulfide bonds can be adapted for expression of the CaNCR peptide variants in E. coli include those disclosed in Kuddus, Biotechnol Prog 233:1520-1528 (2017); Kiedzierska, Protein Expr Purif 60:82-88 (2008); Chang, Amino Acids 47:579-587 (2015); Buchko, Protein Science 27:1611-1623 (2018); Marques, J Appl Microbiol 106:1640-1648 (2008); and Pazgier, Protein Expr Pur 49:1-8 (2006).

In general, the promoter and polyadenylation region are selected based on their operability in a given bacterial, yeast, or fungal host. For example, the AOX1 or AOX2 promoters of Pichia can be used in conjunction with the AOX1, AOX2, p40, or p76 polyadenylation sequences of Pichia to express a heterologous protein such as a CaNCR peptide variant. Both the AOX1 and AOX2 promoters are particularly useful in Pichia as both promoters provide for abundant expression of the linked heterologous gene when induced by addition of methanol to the growth medium. The use of these Pichia promoters and polyadenylation sequences is described in U.S. Pat. No. 4,855,231, which is expressly incorporated herein by reference in its entirety. Similarly, the Hansuela MOX, DHAS, or FMDH promoters can be used to express heterologous peptides such as CaNCR peptide variants in Hansuela. The MOX, DHAS, or FMDH promoters are particularly useful in Hansuela as these promoters provide for abundant expression of the linked heterologous gene when induced by addition of methanol to the growth medium. The use of the MOX and DHAS promoters in Hansuela is described in U.S. Pat. No. 5,741,672, while the use of the FMDH promoter in Hansuela is described in U.S. Pat. No. 5,389,525, each of which is expressly incorporated herein by reference in its entirety. For Kluveromyces, a Lactase promoter and polyadenylation sequence can be used to express heterologous genes such as CaNCR peptide variants. Expression of heterologous genes that are operably linked to the Lactase promoter and polyadenylation sequence is achieved by growing Kluveromyces in the presence of galactose. The use of the Lactase promoter and polyadenylation sequences in Kluveromyces is described in U.S. Pat. No. 6,602,682, which is expressly incorporated herein by reference in its entirety.

Yeast, bacterial, or fungal expression vectors that provide for secretion of heterologous proteins such as a CaNCR peptide variant into the growth medium by transformed yeast or fungi are also contemplated. Secretion of the mature CaNCR peptide variant is typically achieved by operable linkage of a signal peptide sequence or a signal peptide and propeptide sequence to the mature CaNCR peptide variant- or peptide-encoding sequence. Examples of useful signal peptides for secretion of heterologous proteins in yeast include but are not limited to an alpha-factor signal peptide, an invertase signal peptide, and a PHOl signal peptide, all of which are derived from yeast. The alpha-factor signal peptide is typically derived from Saccharomyces, Kluveromyces, or Candida, while the PHOl signal peptide is derived from Pichia.

A particularly useful signal peptide sequence or signal peptide and propeptide sequence for secretion of proteins in yeast is derived from the S. cerevisiae alpha-factor, and is described in U.S. Pat. Nos. 4,546,082, 4,588,684, 4,870,008, and 5,602,034, each of which is incorporated herein by reference in its entirety. The S. cerevisiae alpha-factor signal peptide and propeptide sequence consist of amino acids 1-83 of the primary, unprocessed translation product of the S. cerevisiae alpha mating factor gene (GenBank Accession Number: P01149). In certain embodiments, the signal peptide sequence of the alpha-mating factor comprising amino acids 1 to about 19 to 23 of the alpha-mating factor proprotein can be directly linked to the N-terminus of the mature CaNCR peptide variant to provide for secretion of mature CaNCR peptide variant. In this case, the signal peptide is cleaved from the mature CaNCR peptide variant in the course of the secretion process. Alternatively, the signal peptide and propeptide of the alpha mating factor can be operably linked to the mature CaNCR peptide variant encoding sequence via a cleavage site sequence. This cleavage site sequence can comprise a variety of sequences that provide for proteolytic processing of the leader sequence and gene of interest.

In the native S. cerevisiae alpha mating factor gene the s cleavage site sequence corresponds to amino acid residues 84-89 and is represented by the sequence Lys84-Arg85-Glu86-Ala87-Glu88-Ala 89 (SEQ ID NO: 49). The sequence Lys-Arg corresponds to a KEX2 protease recognition site while the Glu-Ala-Glu-Ala sequence corresponds to a duplicated dipeptidylaminopeptidase or STE13 recognition site. In certain embodiments, a DNA fragment encoding the 89 amino acid S. cerevisiae alpha factor signal, propeptide coding region, and entire native spacer coding region (i.e., the N-terminal 89 amino acid residues of the alpha mating factor precursor protein containing both the Lys-Arg KEX2 protease cleavage site at residues 84 and 85 as well as the Glu-Ala-Glu-Ala dipeptidylaminopeptidase or STE13 recognition site at residues 86-89) is operably linked to the sequence encoding the mature CaNCR peptide variant.

When the N-terminal 89 amino acids of the alpha mating factor precursor protein are fused to the N-terminus of a heterologous protein such as a CaNCR peptide variant, the propeptide sequence is typically dissociated from the heterologous protein via the cleavage by endogenous yeast proteases at either the KEX2 or STE13 recognition sites. In other embodiments, a DNA fragment encoding the smaller 85 amino acid Saccharomyces cerevisiae alpha factor signal peptide, propeptide, and KEX2 spacer element (i.e., the N-terminal 85 amino acid residues of the alpha mating factor precursor protein containing just the Lys-Arg KEX2 protease cleavage site at residues 84 and 85) is operably linked to the sequence encoding the mature CaNCR peptide variant. When the N-terminal 85 amino acids of the alpha mating factor precursor protein are fused to the N-terminus of a heterologous protein such as a CaNCR peptide variant, the propeptide sequence is typically dissociated from the heterologous protein via cleavage by endogenous yeast proteases at the KEX2 recognition site. The CaNCR peptide variant can thus be expressed without the glu-ala repeats.

To obtain transformed yeast that express CaNCR peptide variants, the yeast a CaNCR peptide variant expression cassettes (e.g., yeast promoter, yeast signal peptide encoding sequence, mature CaNCR peptide variant sequence, and polyadenylation sequence) are typically combined with other sequences that provide for selection of transformed yeast. Examples of useful selectable marker genes include genes encoding a ADE protein, a HIS5 protein, a HIS4 protein, a LEU2 protein, a URA3 protein, ARG4 protein, a TRP1 protein, a LYS2 protein, a protein conferring resistance to a bleomycin or phleomycin antibiotic, a protein conferring resistance to chloramphenicol, a protein conferring resistance to G418 or geneticin, a protein conferring resistance to hygromycin, a protein conferring resistance to methotrexate, an a AR04-OFP protein, and a FZF1-4 protein. Similar electable marker cassettes that confer resistance to antibiotics or rescue auxotrophic traits can be used in bacterial or fungal systems.

DNA molecules comprising the yeast CaNCR peptide variant expression cassettes and selectable marker genes are introduced into yeast cells by techniques such as transfection into yeast spheroplasts or electroporation. In certain embodiments, the DNA molecules comprising the yeast CaNCR peptide variant expression cassettes and selectable marker genes are introduced as linear DNA fragments that are integrated into the genome of the transformed yeast host cell. Integration can occur at random sites in the yeast host cell genome or at specific sites in the yeast host cell genome. Integration at specific sites in the yeast host cell genome is typically accomplished by homologous recombination between sequences contained in the expression vector and sequences in the yeast host cell genome. Homologous recombination is typically accomplished by linearizing the expression vector within the homologous sequence (for example, within the AOX1 promoter sequence of a Pichia expression vector when integrating the expression vector into the endogenous AOX1 gene in the Pichia host cell). In other embodiments, the yeast expression cassettes can also comprise additional sequences such as autonomous replication sequences (ARS) that provide for the replication of DNA containing the expression cassette as an extrachromosomal (non-integrated) element. Such extra-chromosomal elements are typically maintained in yeast cells by continuous selection for the presence of the linked selectable marker gene. Yeast artificial chromosomes (YACs) containing sequences that provide for replication and mitotic transmission are another type of vector that can be used to maintain the DNA construct in a yeast host.

Also provided are antimicrobial compositions for agricultural, pharmaceutical, or veterinary use comprising an antimicrobial plant or antimicrobial human or veterinary, pathogenic microbe inhibitory amount (“antimicrobial effective amount”) of one or more the present isolated, purified antimicrobial CaNCR peptide variants, or biologically functional equivalents thereof. Such compositions can comprise one, or any combination of, CaNCR peptide variants disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, CaNCR peptide variant-susceptible pathogenic microbes associated with plant, human or animal microbial infections. Such antimicrobial compositions can be formulated for topical administration, and applied topically to either plants, the plant environment (including soil), or humans or animals.

Agricultural compositions comprising any of the present CaNCR peptide variant molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present CaNCR peptide variants with other pesticidally active substances, fertilizers, and/or growth regulators, etc., in the form of finished formulations or tank mixes.

Whether alone or in combination with other active agents, the present antimicrobial CaNCR peptide variants can be applied at a concentration in the range of from about 0.1 μg ml to about 100 mg ml, or from about 5 μg ml to about 5 mg ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, NaCl in the range of from about 1 mM to about 1 M, or about 10 mM to about 100 mM, can be added. In certain embodiments, CaNCR variant peptides provided herein (e.g., including SEQ ID NO: 11) can exhibit improved antimicrobial activity in comparison to control CaNCR peptides (e.g. SEQ ID NO: 9) in the presence of cations at concentrations that inhibit CaNCR peptide activity (e.g., Na+ at 100 mM or more).

Numerous conventional microbial antibiotics and chemical fungicides with which the present CaNCR peptide variants can be combined are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorous compounds. In addition, azole, triazole, and/or echinocandin fungicides can also be used. Other active ingredients that can be formulated in combination with the present antimicrobial peptides include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial CaNCR peptide variants can be formulated.

Agriculturally useful antimicrobial compositions encompassed herein also include those in the form of host cells, such as bacterial and microbial cells, capable of the producing the CaNCR peptide variants and proteins, and which can colonize plants, including roots, shoots, leaves, or other parts of plants. The term “plant-colonizing microorganism” is used herein to refer to a microorganism that is capable of colonizing the any part of the plant itself and/or the plant environment, including, and which can express the present CaNCR peptide variant antimicrobial peptides in the plant and/or the plant environment. A plant-colonizing microorganism is one that can exist in symbiotic or non-detrimental relationship with a plant in the plant environment. U.S. Pat. No. 5,229,112, which is incorporated herein by reference in its entirety, discloses a variety of plant-colonizing microorganisms that can be engineered to express antimicrobial proteins, and methods of use thereof, applicable to the CaNCR peptide variant antimicrobial peptides disclosed herein. Plant-colonizing microorganisms expressing the presently disclosed CaNCR peptide variant antimicrobial peptides useful in inhibiting microbial growth in plants include bacteria selected from the group consisting of Bacillus spp. including but not limited to Bacillus thuringiensis, Bacillus israelensis, and Bacillus subtilis, Candidatus Liberibacter asiaticus; Pseudomonas spp.; Arthrobacter spp., Azospirillum spp., Clavibacter spp., Escherichia spp.; Agrobacterium spp., for example A. radiobacter, Rhizobium spp., Erwinia spp. Azotobacter spp., Azospirillum spp., Klebsiella spp., Alcaligenes spp., Rhizobacterium spp., Xanthomonas spp., Ralstonia spp. and Flavobacterium spp., In certain embodiments, the microorganism is a yeast selected from the group consisting of Saccharomyces cerevisiae, Pichia pastoris, and Pichia methanolica. In certain embodiments, the plant-colonizing microorganism can be an endophytic bacteria or microbe.

When applying the present CaNCR peptide variant molecules to the rhizosphere, rhizosphere-colonizing bacteria from the genus Pseudomonas are particularly useful, especially the fluorescent pseudomonads, e.g., Pseudomonas fluorescens, which is especially competitive in the plant rhizosphere and in colonizing the surface of the plant roots in large numbers. Examples of suitable phylloplane (leaf) colonizing bacteria are P. putida, P. syringae, and Erwinia species.

The antimicrobial plant-colonizing microorganisms that can express CaNCR peptide variant can be applied directly to the plant, e.g., to the surface of leaves, buds, roots, shoots, floral parts, seeds, etc., or to the soil. When used as a seed coating, the plant-colonizing microorganisms that can express CaNCR peptide variant are applied to the plant seed prior to planting. The determination of an antimicrobial effective amount of plant-colonizing microorganisms used for a particular plant can be empirically determined and will depend on such factors as the plant species, the microbial pathogen, method of planting, and the soil type, (e.g., pH, organic matter content, moisture content). At least one, 10 or 100 plant-colonizing microorganism(s) containing DNA encoding the CaNCR peptide variant antimicrobial peptides disclosed herein is sufficient to control microbial pathogens because it or they can grow into a colony of clones of sufficient number to express antimicrobial amounts of the CaNCR peptide variant. However, in practice, due to varying environmental factors which can affect the survival and propagation of the microorganism, a sufficient number of plant colonizing microorganisms should be provided in the seed, plant or plant environment (e.g., roots or foliage) to assure survival and/or proliferation. For example, application of 10³ to 10¹⁰ bacteria or yeasts per seed can be sufficient to insure colonization on the surface of the roots by the microorganism. In certain embodiments, it is sufficient to dose the plant or plant environment with enough bacteria or other plant-colonizing microorganism to maintain a population that expresses 100 to 250 nanograms of the CaNCR peptide variant per plant. For example, 10⁵ to 108 bacteria per square centimeter of plant surface can be adequate to control microbial infection. In certain embodiments, at least about 5 or 10 nanograms to about 100, 200, 500, or 1,000 nanograms, of a CaNCR peptide variant or CaNCR peptide variant multimer can be sufficient to control microbial damage to plants.

Compositions containing the plant colonizing microorganisms that express the CaNCR peptide variant can be prepared by formulating the biologically active microorganism with adjuvants, diluents, carriers, etc., to provide compositions in the form of finely divided particulate solids, granules, pellets, wettable powders, dusts, aqueous suspensions, dispersions, or emulsions. Illustrative of suitable carrier vehicles are solvents, e.g., water or organic solvents, and finely divided solids, e.g., kaolin, chalk, calcium carbonate, talc, silicates, and gypsum. In certain embodiments, plant-colonizing microorganisms that express the CaNCR peptide variant can also be in encapsulated form, e.g., the plant-colonizing microorganisms can be encapsulated within shell walls of polymer, gelatin, lipid, and the like. Other formulation aids such as, for example, emulsifiers, dispersants, surfactants, wetting agents, anti-foam agents, and anti-freeze agents, can be incorporated into the antimicrobial compositions, especially if such compositions will be stored for any period of time prior to use.

In addition to the plant-colonizing microorganisms that express CaNCR peptide variant, the compositions provided herein can additionally contain other known biologically active agents, such as, for example, a fungicide, herbicide, or insecticide. In addition, two or more plant-colonizing microorganisms that express either a different or the same CaNCR peptide variant can be combined.

The application of antimicrobial compositions containing the genetically engineered plant-colonizing microorganisms that can express CaNCR peptide variant as the active agent can be carried out by conventional techniques utilizing, for example, spreaders, power dusters, boom and hand sprayers, spry dusters, and granular applicators.

The compositions provided herein can be applied in an antimicrobial effective amount, which will vary depending on such factors as, for example, the specific fungal pathogen to be controlled, the specific plant (and plant part or soil) to be treated, and the method of applying the compositions that comprise CaNCR peptide variants and proteins.

CaNCR peptide variants and biologically functional equivalents, as well as transgenic or genetically edited plants or microorganisms expressing those proteins, can be used to inhibit the growth of a wide variety of susceptible microbes in plants. In certain embodiments, growth of microbes in the following genera or species can be inhibited: Alternaria (e.g., Alternaria brassicicola; Alternaria solani); Ascochyta (e.g., Ascochyta pisi); Aspergillus (e.g., Aspergillus flavus; Aspergillus fumigatus); Botrytis (e.g., Botrytis cinerea); Cercospora (e.g., Cercospora kikuchii; Cercospora zeae-maydis); Colletotrichum (e.g., Colletotrichum lindemuthianum); Diplodia (e.g., Diplodia maydis); Erysiphe (e.g., Erysiphe graminis f. sp. graminis, Erysiphe graminis f. sp. hordei); Fusarium (e.g., Fusarium nivale; Fusarium oxysporum; Fusarium graminearum; Fusarium culmorum; Fusarium solani; Fusarium moniliforme; Fusarium roseum); Gaeumannomyces (e.g., Gaeumannomyces graminis f. sp. tritici); Helminthosporium (e.g., Helminthosporium turcicum; Helminthosporium carbonum; Helminthosporium maydis); Macrophomina (e.g., Macrophomina phaseolina; Magnaporthe grisea); Nectria (e.g., Nectria haematococca); Peronospora (e.g., Peronospora manshurica; Peronospora tabacina); Phakopsora (e.g., Phakopsora pachyrhizi); Phoma (e.g., Phoma betae); Phymatotrichum (e.g., Phymatotrichum omnivorum); Phytophthora (e.g., Phytophthora cinnamomi; Phytophthora cactorum; Phytophthora phaseoli; Phytophthora parasitica; Phytophthora citrophthora; Phytophthora sojae; Phytophthora infestans); Plasmopara (e.g., Plasmopara viticola); Podosphaera (e.g., Podosphaera leucotricha); Puccinia (e.g., Puccinia sorghi; Puccinia striiformis; Puccinia graminis f. sp. tritici; Puccinia asparagi; Puccinia recondita; Puccinia arachidis); Pythium (e.g., Pythium aphanidermatum; Pythium ultimum); Pyrenophora (e.g., Pyrenophora tritici-repentens); Pyricularia (e.g., Pyricularia oryzae); Rhizoctonia (e.g., Rhizoctonia solani; Rhizoctonia cerealis); Sclerotium (e.g., Sclerotium rolfsii); Sclerotinia (e.g., Sclerotinia sclerotiorum); Septoria (e.g., Septoria lycopersici; Septoria glycines; Septoria nodorum; Septoria tritici); Thielaviopsis (e.g., Thielaviopsis basicola); Uncinula (e.g., Uncinula necator); Venturia (e.g., Venturia inaequalis); and Verticillium (e.g., Verticillium dahliae; Verticillium albo-atrum).

Pharmaceutical or veterinary compositions that comprise an antimicrobial effective amount of a CaNCR peptide variant, or biologically functional equivalents thereof and a pharmaceutically acceptable carrier are also provided. Such pharmaceutical or veterinary compositions can be used for inhibiting the growth of, or killing, susceptible pathogenic microbes that infect humans or animals, i.e., treating such fungal infections by administering to a patient or other subject in need thereof. In certain embodiments, compositions comprising CaNCR peptide variants and proteins, and biologically functional equivalents thereof, can be formulated by methods such as those described in Remington: The Science and Practice of Pharmacy (2005), 21st Edition, University of the Sciences in Philadelphia, Lippincott Williams & Wilkins. In certain embodiments, the compositions can contain CaNCR peptide variants and proteins, and various combinations thereof, at concentrations in the range of from about 0.1 μg per ml to about 100 mg per ml, or about 5 μg per ml to about 5 mg per ml, at a pH in the range of from about 3.0 to about 9.0. Such compositions can be buffered using, for example, phosphate buffers at a concentration of about 1 mM to about 1 M, about 10 mM to about 100 mM, or about 15 mM to 50 mM. In the case of low buffer concentrations, a salt can be added to increase the ionic strength. In certain embodiments, NaCl in the range of about 1 mM to about 1 M, or about 10 mM to about 100 mM, can be added.

The CaNCR peptide variants can be formulated alone, in any combination with one another, and either of these can additionally be formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine.

Administration of the compositions that comprise CaNCR peptide variant to a human or animal subject in need thereof can be accomplished via a variety of routes that include topical application.

### Embodiments

1. A peptide comprising the amino acid sequence of: (a) SEQ ID NO: 1, optionally wherein the C-terminal amino acid residue is amidated; (b) SEQ ID NO: 5, wherein the peptide does not comprise the amino acid sequence of SEQ ID NO: 9 and optionally wherein the C-terminal amino acid residue is amidated; or (c) SEQ ID NO: 42, wherein said peptide comprising SEQ ID NO: 1, SEQ ID NO: 5, or SEQ ID NOs: 42 is 13 to 18, 25, or 30 amino acid residues in length and wherein said peptide contains only the two cysteine residues set forth in SEQ ID NO: 1, SEQ ID NO: 5, or SEQ ID NO: 42.

2. The peptide of embodiment 1, wherein the peptide comprises the amino acid sequence of SEQ ID NO: 2, SEQ ID NO: 3, or SEQ ID NO: 4.

3. The peptide of embodiment 1 or 2, wherein the peptide comprises SEQ ID NO: 11, SEQ ID NO: 12, SEQ ID NO: 13, SEQ ID NO: 21, SEQ ID NO: 22, SEQ ID NO: 23, SEQ ID NO: 29, SEQ ID NO: 30, SEQ ID NO: 34 or SEQ ID NO: 35.

4. The peptide of embodiment 1, 2, or 3, wherein the peptide comprises the amino acid sequence of SEQ ID NO: 6, SEQ ID NO: 7, or SEQ ID NO: 8.

5. The peptide of embodiment 4, wherein said peptide comprises the amino acid sequence of SEQ ID NO: 10, SEQ ID NO: 17, SEQ ID NO: 18, SEQ ID NO: 19, SEQ ID NO: 20, SEQ ID NO: 25, SEQ ID NO: 26, SEQ ID NO: 27, SEQ ID NO: 28, SEQ ID NO: 31, SEQ ID NO: 32, or SEQ ID NO: 33.

6. The peptide of any one of embodiments 1 to 5, wherein said peptide comprises: (a) at least one non-conservative substitution of a corresponding amino acid residue in SEQ ID NOs: 9, 16, 24, 36, 37, 38, 39, or 40, optionally wherein: (i) the non-conservative substitution comprises substitution of a hydrophobic, neutral polar, or acidic amino acid residue with a basic amino acid residue; (ii) the non-conservative substitution comprises substitution of an acidic amino acid residue with a basic amino acid residue, tyrosine, phenylalanine, or tryptophan; (iii) the non-conservative substitution comprises substitution of a neutral polar amino acid residue with a phenylalanine or tryptophan residue; or (i) a non-conservative amino acid substitution set forth in in FIG. 1.

7. The peptide of any one of embodiments 1 to 6, wherein said peptide comprises 1 to 8 additional amino acids at the amino (N) terminus, at the carboxy (C) terminus, at both the N-terminus and the C-terminus of the peptide SEQ ID NO: 1; optionally wherein the peptide comprises or further comprises an alanine residue at its N-terminus.

8. The peptide of any one of embodiments 1 to 7, wherein said peptide exhibits increased antifungal activity in the presence of 100 mM sodium chloride in comparison to antifungal activity of a control peptide comprising SEQ ID NOs: 9, 16, 24, 36 in the presence of 100 mM sodium chloride.

9. The peptide of any one of embodiments 1 to 8, wherein said peptide does not contain the final six C-terminal amino acid residues of SEQ ID NOs: 37, 38, 39, 40, or a conservatively substituted variant thereof.

10. The peptide of any one of embodiments 1 to 9, wherein the C-terminal residue is the C-terminal cysteine residue of SEQ ID NO: 1 or SEQ ID NO: 5, and optionally wherein said C-terminal cysteine residue is amidated.

11. A composition comprising the peptide of any of one embodiments 1 to 10 and an agriculturally, pharmaceutically, or veterinary-practicably acceptable carrier, diluent, or excipient.

12. The composition of embodiment 11, wherein the peptide is provided at a concentration of about 0.1, 0.5, 1.0, or 5 μg/ml to about 1, 5, 20, 50, or 100 mg/ml or at a concentration of about 0.1, 0.5, 1.0, or 5 pg/gram to about 1, 5, 20, 50, or 100 mg/gram and optionally wherein the composition comprises a sodium salt at a concentration of at least 100 mM and/or a calcium salt at a concentration of at least 2 mM.

13. A method for preventing or reducing crop damage by a plant pathogenic microbe comprising the step of contacting a plant, a plant seed, or other part of said plant with an effective amount of the composition of embodiment 11 or embodiment 12.

14. The method of embodiment 13, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochlobolus sp., Helminthosporium sp., Diplodia sp. or Stenocarpella sp.

15. A medical device comprising the device and the composition of embodiment 11 or 12, wherein the device comprises at least one surface that is topically coated and/or impregnated with the composition.

16. The medical device of embodiment 15, wherein said device is a stent, a catheter, a contact lens, a condom, a patch, or a diaphragm.

17. A method for treating, preventing, or inhibiting a microbial infection in a subject in need thereof comprising administering to said subject an effective amount of the composition of embodiment 11 or 12.

18. The method of embodiment 17, wherein said administration comprises topical, enteral, parenteral, and/or intravenous introduction of the composition.

19. The method of embodiment 17 or 18, wherein the subject is a human, livestock, poultry, fish, or a companion animal.

20. The method of embodiment 17, 18, or 19, wherein the microbial infection is of a mucosal membrane, eye, skin, and/or a nail and the composition is applied to the mucosal membrane, eye, skin, and/or nail.

21. The method of any one of embodiments 17 to 20, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum Epidermophyton floccosum, and Microsporum gypseum.

22. The method of any one of embodiments 17 to 20, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

23. The method of any one of embodiments 17 to 20, wherein the microbial infection is by a Candida species and wherein the Candida species is Candida albicans (C. albicans), C. auris, C. glabrata, C parasilosis, C. tropicalis, or C. krusei.

24. The composition of embodiment 11 or 12 for use in a method of treating, preventing, or inhibiting microbial infection in a subject in need thereof.

25. The composition of embodiment 24, wherein the subject is a human, livestock, poultry, fish, or a companion animal.

26. A plant part that is at least partly coated with the composition of embodiment 11 or 12.

27. The plant part of embodiment 26, wherein the part is a seed and the seed is optionally a corn, soybean, wheat, rice, cotton, Brassica sp., or tomato seed.

28. The plant part of embodiment 26 or 27, wherein the plant part is a fruit, vegetable, or flower.

29. A recombinant polynucleotide comprising a polynucleotide encoding a peptide comprising the peptide of any one of embodiments 1 to 10, wherein the polynucleotide encoding the first antimicrobial peptide is operably linked to a polynucleotide comprising a promoter which is heterologous to the polynucleotide encoding the first antimicrobial peptide, optionally wherein any amino acid substitution in said sequence increases or maintains the net positive charge and/or increases or maintains hydrophobicity of the peptide.

30. The recombinant polynucleotide of embodiment 29, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.

31. The recombinant polynucleotide of embodiment 29 or 30, wherein the polynucleotide encoding the first antimicrobial peptide is inserted into a heterologous nuclear or plastid genome of a cell and operably linked to an endogenous promoter located in the heterologous nuclear or plastid genome.

32. A plant nuclear or plastid genome comprising a polynucleotide encoding a peptide comprising the peptide of any one of embodiments 1 to 10, wherein the polynucleotide is heterologous to the nuclear or plastid genome and wherein the polynucleotide is operably linked to an endogenous promoter of the nuclear or plastid genome.

33. A cell comprising the recombinant polynucleotide of any one of embodiments 29 to 31 or the genome of embodiment 32, wherein the cell is optionally a bacterial, yeast, or plant cell.

34. A plant comprising the recombinant polynucleotide of any one of embodiments 29 to 31 or the genome of embodiment 32.

35. A plant part of the plant of embodiment 34, wherein the plant part comprises the recombinant polynucleotide or genome, optionally wherein the plant part is a seed, stem, leaf, root, tuber, flower, or fruit.

36. A method for producing plant seed that provides plants resistant to infection by a plant pathogenic microbe that comprises the steps of (i) selfing or crossing the plant of embodiment 34; and (ii) harvesting seed that comprises the recombinant polynucleotide of the plant from the self or cross, thereby producing plant seed that provide plants resistant to infection by a plant pathogenic microbe.

37. A method of making the composition of any one of claims 11 or 12, comprising the step of combining the peptide with an agriculturally, pharmaceutically, or veterinary-practicably acceptable carrier, diluent, or excipient.

The method of claim 37, further comprising the step of obtaining the peptide by vitro synthesis.

The method of claim 37, further comprising obtaining the peptide from a culture of a bacterial or yeast cell of claim 33.

### EXAMPLES

### Example 1. Synthesis and Purification of Peptides

The chemically synthesized NCR13_V7 and NCR13_V9 peptides with 80-85% purity were obtained from Biomatik Inc, Canada. Each peptide was purified homogeneity using a linear gradient of acetonitrile/water mixture in a C-18 reverse phase HPLC (Agilent Technologies, CA, USA), with a C18 column. HPLC fractions containing each peptide were lyophilized and resuspended in nuclease-free water. The concentration of each peptide was determined using the BCA assay performed according to the manufacturer's protocol (Thermo-Fisher Scientific). The purity and size of each peptide were verified by sodium dodecyl sulfate-polyacrylamide gel electrophoresis (SDS-PAGE). Mass spectrometric analysis was performed at the Proteomics and Mass Spectrometry Facility at the Donald Danforth Plant Science Center to confirm the correct mass for each peptide. Amino acid sequences, net charge and hydrophobicity of NCR13_V7 and NCR13_V9 are provided in Table 11.

TABLE 11
Amino acid sequences, net charge and
hydrophobicity of the designed peptides.

| Pep- | Sequence | Net | Hydro- |
|---|---|---|---|
| tide | (SEQ ID NO) | Charge | phobicity |
| NCR13_V7 | KKFACRKPKVPKC-NH₂ | +6 | 38.5% |
| | (SEQ ID NO: 9) | | |
| NCR13_V9 | KKFWCRKPKWPKC-NH₂ | +6 | 38.5% |
| | (SEQ ID NO: 11) | | |

### Example 2. Determination of the Minimum Inhibitory Concentration (MIC) of NCR13_V7 and NCR13_V9 in Absence and Presence of Cations

The fungal strain of Botrytis cinerea T-4 was cultured on V8 agar plates. Fungal spores were harvested by flooding the fungal growth plates with sterile water. The spore suspension was filtered through two layers of Miracloth, centrifuged at 13,600 rpm for 1 min, washed, and re-suspended in low-salt Synthetic Fungal Medium (SFM) (U.S. Pat. No. 6,316,407, incorporated herein by reference in its entirety). The spore suspension was adjusted to the desired spore count using a hemocytometer.

Antifungal activity of NCR13_V7 (SEQ ID NO: 9) and NCR13_V9 (SEQ ID NO: 11) was assessed at different concentrations using a 2-fold dilution series of each peptide. Antifungal activity of each peptide was determined spectrophotometrically using the 96-well plate assay (Kereszt et al., Front. Plant Sci. 9, 1026 (2018)). Around 45 μL of each peptide at different concentrations was added to each well of the microtiter plate containing 45 μL of ˜10⁵ spores/ml spore suspension. The quantitative fungal growth inhibition was determined by measuring the absorbance at 595 nm using a (Tecan Infinite M200 ProTecan Systems Inc., San Jose, CA) microplate reader after 48 h. The fungal cell viability was determined by the resazurin cell viability assay (Tiricz et al., Appl. Environ. Microbiol. 79, 6737-6746 (2013); Balogh et al., Acta Microbiol. Immunol. Hung. 61, 229-239 (2014)). After incubation of the pathogen/peptide mixture for 48 h, 10 μl of 0.1% resazurin solution was added to each well. After incubation of the mixture overnight, a change in the color of the resazurin dye from blue to pink or colorless indicated the presence of live fungal cells. The MIC for each peptide was the lowest concentration of each peptide at which no change in blue color occurred. Using this protocol, the MIC values of NCR13_V7 and NCR13_V9 were also determined in presence of 100 mM NaCl.

The MIC values of NCR13_V7 and NCR13_V9 were determined to be 3 μM and 1.5 μM against B. cinerea in SFM. NCR13_V9 exhibits 2-fold higher antifungal activity against this pathogen in SFM (Table 12). It has been hypothesized that the presence of cations significantly weakens the electrostatic interactions between a positively charged peptides and negatively charged fungal membranes (7). We therefore evaluated the antifungal activity of these peptides in SFM supplemented with 100 mM NaCl (Table 12).

TABLE 12
Antifungal activity of NCR13_V7 and
NCR13_V9 against B. cinerea

| | | MIC (μM) | MIC (μM) in SFM + |
|---|---|---|---|
| Peptide | SEQ ID NO: | in SFM | 100 mM NaCl |
| NCR13_V7 | SEQ ID NO: 9 | 3 | 6 |
| NCR13_V9 | SEQ ID NO: 11 | 1.5 | 1.5-3 |

### Example 3. NCR13_V9 is More Effective in Conferring Resistance to Gray Mold than NCR13_V7 in a Semi-In Planta Assay

Nicotiana benthamiana Nbl plants were grown in a controlled environment growth chamber for two weeks under controlled conditions. Detached leaves were placed on petri dishes and were used for semi-in planta antifungal activity assays as described previously (Li et al. Mol Plant Microbe Interact 32, 1649-1664 (2019); Velivelli et al., Proceedings of the National Academy of Sciences. 117(27)16043-16054 (2020)). A 10 μl aliquot of NCR13_V7 and NCR13_V9 at different concentrations (1.5, 3.0 and 6 μM) was spotted onto the detached leaves. After spotting each peptide at appropriate position, 10 μL of B. cinerea spores (˜2.5×10⁵ spores/mL suspended in 2×SFM buffer) was added to each peptide spot. The control inoculum contained fungal spores alone in 1×SFM with no peptide. The petri dishes were placed into Ziploc Weather-Shield plastic boxes containing wet paper towels to maintain high humidity. Following incubation at room temperature for 48 h, leaves containing disease lesions were photographed in white light. High-resolution fluorescence images were also taken using CropReporter (PhenoVation, Wageningen, Netherlands). The quantification of plant health/stress was carried out using the calculated F_{V}/F_{M} (maximum quantum yield of photosystem II) images showing the efficiency of photosynthesis in false colors.

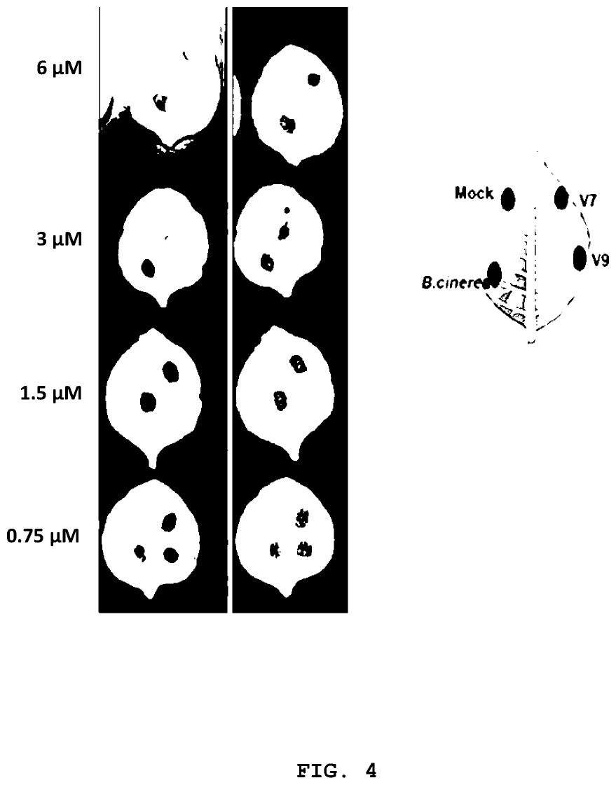

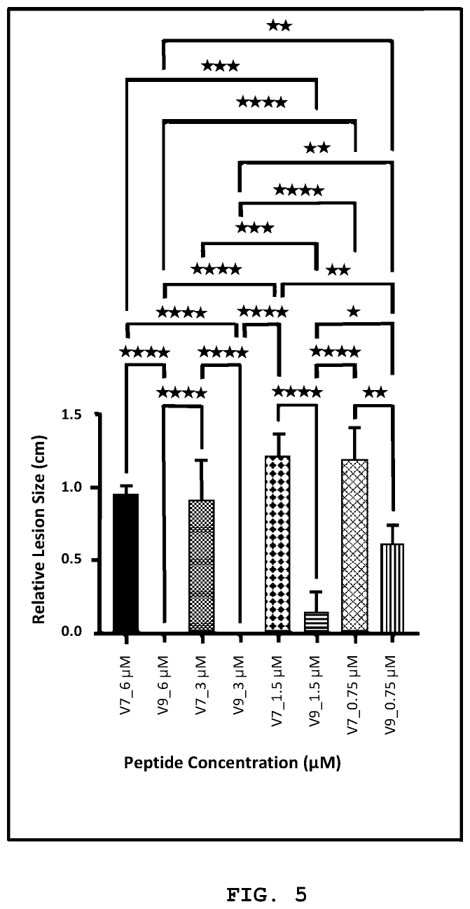

NCR13_V7 and NCR13_V9 were also evaluated for their ability to confer resistance to gray mold on detached N. benthamiana leaves (FIGS. 4 and 5). Three concentrations (0.75, 1.5, 3 and 6 μM) of each peptide were tested for antifungal activity. At all concentrations, NCR13_V9 reduced the lesion sizes greater than NCR13_V7. We observed a significant decrease in disease symptoms at lower concentrations of peptide used in the experiment.

### Example 4. Alanine Scanning Mutagenesis of the NCR13 Peptide

The chemically synthesized NCR13 and the variants NCR13_AlaV1-V5 (SEQ ID NO: 43-47; Table 10) were obtained from Biomatik Inc, Canada. Each peptide was purified to homogeneity using a linear gradient of acetonitrile/water mixture in a C-18 reverse phase HPLC (Agilent Technologies, CA, USA), with a C18 column. HPLC fractions containing each peptide were lyophilized and resuspended in nuclease-free water. The concentration of each peptide was determined using the BCA assay performed according to the manufacturer's protocol (Thermo-Fisher Scientific). The purity and size of each peptide were verified by sodium dodecyl sulfate-polyacrylamide gel electrophoresis (SDS-PAGE). Mass spectrometric analysis was performed at the Proteomics and Mass Spectrometry Facility at the Donald Danforth Plant Science Center to confirm the correct mass for each peptide.

Antifungal activity of NCR13_AlaV1-V5 was assessed at different concentrations using a 2-fold dilution series of each peptide. Antifungal activity of each peptide was determined spectrophotometrically using the 96-well plate assay. Around 45 μL of each peptide at different concentrations was added to each well of the microtiter plate containing 45 μL of ˜105 spores/ml spore suspension. The quantitative fungal growth inhibition was determined by measuring the absorbance at 595 nm using a (Tecan Infinite M200 ProTecan Systems Inc., San Jose, CA) microplate reader after 48 h. The fungal cell viability was determined by the resazurin cell viability assay. After incubation of the pathogen/peptide mixture for 48 h, 10 μl of 0.1% resazurin solution was added to each well. After incubation of the mixture overnight, a change in the color of the resazurin dye from blue to pink or colorless indicated the presence of live fungal cells. The MIC for each peptide was the lowest concentration of each peptide at which no change in blue color occurred. Results are summarized below in Table 13.

TABLE 13
Antifungal activity of NCR13 and NCR13_V1-V6
was determined against Botrytis cinerea and
Fusarium graminearum

| | | MIC | MIC |
|---|---|---|---|
| | | (μM) | (μM) |
| Pep- | Sequence | Botrytis | Fusarium |
| tide | (SEQ ID NO) | cinerea | graminearum |
| NCR13 | TKPCQSDKDCKKFACRKPK | 3 | 6 |
| | VPKCINGFCKCVR | | |
| | (SEQ ID NO: 38) | | |
| NCR13_ | TKPCAAAAACKKFACRKPK | 6 | 6 |
| AlaV1 | VPKCINGFCKCVR | | |
| | (SEQ ID NO: 43) | | |
| NCR13_ | TKPCQSDKDCAAAACRKPK | Inactive | Inactive |
| AlaV2 | VPKCINGFCKCVR | | |
| | (SEQ ID NO: 44) | | |
| NCR13_ | TKPCQSDKDCKKFACAAAA | Inactive | Inactive |
| AlaV3 | VPKCINGFCKCVR | | |
| | (SEQ ID NO: 45) | | |
| NCR13_ | TKPCQSDKDCKKFACRKPK | Inactive | 12 |
| AlaV4 | AAACINGFCKCVR | | |
| | (SEQ ID NO: 46) | | |
| NCR13_ | TKPCQSDKDCKKFACRKPK | 12 | 6 |
| AlaV5 | VPKCAAAACKCVR | | |
| | (SEQ ID NO: 47) | | |

In vitro antifungal activity of NCR13 and its alanine scanning variants (NCR13_V1-V5) revealed that the sequence motif KKFACRKPKV (SEQ ID NO: 48) is the major sequence motif responsible for the antifungal activity of this peptide (Table 13). The sequence motif VPK also makes significant contribution the antifungal activity of NCR13 against B. cinerea and F. graminearum. The sequence motif QSDKD and INGF also make minor contributions to the antifungal activity of this peptide against NCR13 (Table 13).

What is claimed is:

1. A peptide comprising the amino acid sequence of: (a) SEQ ID NO: 1, optionally wherein the C-terminal amino acid residue is amidated; (b) SEQ ID NO: 5, wherein the peptide does not comprise the amino acid sequence of SEQ ID NO: 9 and optionally wherein the C-terminal amino acid residue is amidated; or (c) SEQ ID NO: 42, wherein said peptide comprising SEQ ID NO: 1, SEQ ID NO: 5, or SEQ ID NOs: 42 is 13 to 18, 25, or 30 amino acid residues in length and wherein said peptide contains only the two cysteine residues set forth in SEQ ID NO: 1, SEQ ID NO: 5, or SEQ ID NO: 42.

2. The peptide of claim 1, wherein the peptide comprises the amino acid sequence of SEQ ID NO: 2, SEQ ID NO: 3, or SEQ ID NO: 4.

3. The peptide of claim 1, wherein the peptide comprises SEQ ID NO: 11, SEQ ID NO: 12, SEQ ID NO: 13, SEQ ID NO: 21, SEQ ID NO: 22, SEQ ID NO: 23, SEQ ID NO: 29, SEQ ID NO: 30, SEQ ID NO: 34, or SEQ ID NO: 35.

4. The peptide of claim 1, wherein the peptide comprises the amino acid sequence of SEQ ID NO: 6, SEQ ID NO: 7, or SEQ ID NO: 8.

5. The peptide of claim 4, wherein said peptide comprises the amino acid sequence of SEQ ID NO: 10, SEQ ID NO: 17, SEQ ID NO: 18, SEQ ID NO: 19, SEQ ID NO: 20, SEQ ID NO: 25, SEQ ID NO: 26, SEQ ID NO: 27, SEQ ID NO: 28, SEQ ID NO: 31, SEQ ID NO: 32, or SEQ ID NO: 33.

6. The peptide of any one of claims 1 to 5, wherein said peptide comprises: (a) at least one non-conservative substitution of a corresponding amino acid residue in SEQ ID NO: 9, 16, 24, 36, 37, 38, 39, or 40, optionally wherein: (i) the non-conservative substitution comprises substitution of a hydrophobic, neutral polar, or acidic amino acid residue with a basic amino acid residue; (ii) the non-conservative substitution comprises substitution of an acidic amino acid residue with a basic amino acid residue, tyrosine, phenylalanine, or tryptophan; (iii) the non-conservative substitution comprises substitution of a neutral polar amino acid residue with a phenylalanine or tryptophan residue; or (i) a non-conservative amino acid substitution set forth in FIG. 1.

7. The peptide of any one of claims 1 to 5, wherein said peptide comprises 1 to 8 additional amino acids at the amino (N) terminus, at the carboxy (C) terminus, at both the N-terminus and the C-terminus of the peptide SEQ ID NO: 1; optionally wherein the peptide comprises or further comprises an alanine residue at its N-terminus.

8. The peptide of any one of claims 1 to 5, wherein said peptide exhibits increased antifungal activity in the presence of 100 mM sodium chloride in comparison to antifungal activity of a control peptide comprising SEQ ID NO: 9, 16, 24, 36 in the presence of 100 mM sodium chloride.

9. The peptide of any one of claims 1 to 5, wherein said peptide does not contain the final six C-terminal amino acid residues of SEQ ID NO: 37, 38, 39, 40, or a conservatively substituted variant thereof.

10. The peptide of any one of claims 1 to 5, wherein the C-terminal residue is the C-terminal cysteine residue of SEQ ID NO: 1 or SEQ ID NO: 5, and optionally wherein said C-terminal cysteine residue is amidated.

11. A composition comprising the peptide of any of one claims 1 to 5 and an agriculturally, pharmaceutically, or veterinary-practicably acceptable carrier, diluent, or excipient.

12. The composition of claim 11, wherein the peptide is provided at a concentration of about 0.1, 0.5, 1.0, or 5 pg/ml to about 1, 5, 20, 50, or 100 mg/ml or at a concentration of about 0.1, 0.5, 1.0, or 5 pg/gram to about 1, 5, 20, 50, or 100 mg/gram and optionally wherein the composition comprises a sodium salt at a concentration of at least 100 mM and/or a calcium salt at a concentration of at least 2 mM.

13. A method for preventing or reducing crop damage by a plant pathogenic microbe comprising the step of contacting a plant, a plant seed, or other part of said plant with an effective amount of the composition of claim 11 or claim 12.

14. The method of claim 13, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp. or Stenocarpella sp.

15. A medical device comprising the composition of claim 11 or 12, wherein the device comprises at least one surface that is topically coated and/or impregnated with the composition.

16. The medical device of claim 15, wherein said device is a stent, a catheter, a contact lens, a condom, a patch, or a diaphragm.

17. A method for treating, preventing, or inhibiting a microbial infection in a subject in need thereof comprising administering to said subject an effective amount of the composition of claim 11 or 12.

18. The method of claim 17, wherein said administration comprises topical, enteral, parenteral, and/or intravenous introduction of the composition.

19. The method of claim 17, wherein the subject is a human, livestock, poultry, fish, or a companion animal.

20. The method of claim 17, wherein the microbial infection is of a mucosal membrane, eye, skin, and/or a nail and the composition is applied to the mucosal membrane, eye, skin, and/or nail.

21. The method of any one of claims 17 to 20, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

22. The method of any one of claims 17 to 20, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

23. The method of any one of claims 17 to 20, wherein the microbial infection is by a Candida species and wherein the Candida species is Candida albicans (C. albicans), C. auris, C. glabrata, C parasilosis, C. tropicalis, or C. krusei.

24. The composition of claim 11 or 12 for use in a method of treating, preventing, or inhibiting microbial infection in a subject in need thereof.

25. The composition of claim 24, wherein the subject is a human, livestock, poultry, fish, or a companion animal.

26. A plant part that is at least partly coated with the composition of claim 11 or 12.

27. The plant part of claim 26, wherein the part is a seed and the seed is optionally a corn, soybean, wheat, rice, cotton, Brassica sp., or tomato seed.

28. The plant part of claim 26 or 27, wherein the plant part is a fruit, vegetable, or flower.

29. A recombinant polynucleotide comprising a polynucleotide encoding a peptide comprising the peptide of any one of claims 1 to 5, wherein the polynucleotide encoding the first antimicrobial peptide is operably linked to a polynucleotide comprising a promoter which is heterologous to the polynucleotide encoding the first antimicrobial peptide, optionally wherein any amino acid substitution in said sequence increases or maintains the net positive charge and/or increases or maintains hydrophobicity of the peptide.

30. The recombinant polynucleotide of claim 29, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.

31. The recombinant polynucleotide of claim 29, wherein the polynucleotide encoding the first antimicrobial peptide is inserted into a heterologous nuclear or plastid genome of a cell and operably linked to an endogenous promoter located in the heterologous nuclear or plastid genome.

32. A plant nuclear or plastid genome comprising a polynucleotide encoding a peptide comprising the peptide of any one of claims 1 to 5, wherein the polynucleotide is heterologous to the nuclear or plastid genome and wherein the polynucleotide is operably linked to an endogenous promoter of the nuclear or plastid genome.

33. A cell comprising the recombinant polynucleotide of any one of claims 29 to 31 or the genome of claim 32, wherein the cell is optionally a bacterial, yeast, or plant cell.

34. A plant comprising the recombinant polynucleotide of any one of claims 29 to 31 or the genome of claim 32.

35. A plant part of the plant of claim 34, wherein the plant part comprises the recombinant polynucleotide or genome, optionally wherein the plant part is a seed, stem, leaf, root, tuber, flower, or fruit.

36. A method for producing plant seed that provides plants resistant to infection by a plant pathogenic microbe that comprises the steps of (i) selfing or crossing the plant of claim 34; and (ii) harvesting seed that comprises the recombinant polynucleotide of the plant from the self or cross, thereby producing plant seed that provide plants resistant to infection by a plant pathogenic microbe.
